(19) United States
(12) Patent Application Publication
(10) Pub. No.: US 2006/0292586 A1
(43) Pub. Date: Dec. 28, 2006

(54) ID-tag complexes, arrays, and methods of use thereof

(76) Inventors: Gary P. Schroth, San Ramon, CA (US); Kai Qin Lao, Pleasanton, CA (US); Neil A. Straus, Emeryville, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/291,444

(22) Filed: Nov. 30, 2005

Related U.S. Application Data
(60) Provisional application No. 60/637,351, filed on Dec. 17, 2004.

(51) Int. Cl. C12Q 1/68; C12M 3/00

(52) U.S. Cl. 435/6; 435/287.2

(57) ABSTRACT

The present invention relates to the detection of target sequences. Detection can be achieved through the use of ID-tag complexes. These ID-tag complexes are relatively stable in the absence of a target sequence. In the presence of a target sequence, the complexes dissociate and form new complexes or duplexes, which can be purified or eliminated and detected on an ID-tag system.

### PRIORITY

This Application claims priority under 35 U.S.C. §119(e) to U.S. Provisional Patent Application Ser. No. 60/637,351, filed Dec. 17, 2005, herein incorporated by reference in its entirety.

### FIELD

The invention relates to methods and compositions for detection of nucleic acids. Particular embodiments relate to an addressable array system and methods of using the addressable array system to detect nucleic acids.

### INTRODUCTION

Despite considerable progress in transcription and translational profiling with gene and protein microarrays, methods and compositions that continuously monitor gene expression dynamics in cells are in high demand. In addition, current microarray technologies cannot detect low copy number gene products, which often play a prominent role in sensing, signaling and gene regulation. One possible method for achieving this goal is through the use of single-molecule detection.

### SUMMARY

In one aspect an ID tag-complex is provided. The complex comprises a probe section that comprises a probe sequence connected to an ID tag sequence and further connected to a detectable marker; and a probe complement section that comprises a probe complement sequence connected to a first coupling molecule. A portion of the probe sequence and the probe complement sequence are configured to hybridized to one another. The length of the probe sequence is at least 1 nucleotide greater than the length of the probe complement sequence. In some embodiments, the detectable marker is connected to an end of the probe sequence and the ID tag sequence is connected an opposite end of the probe sequence. In some embodiments the probe section comprises an analog probe sequence, such as L-DNA or PNA. In some embodiments the probe sequence will bind to RNA or miRNA. In some embodiments the probe sequence is 5-10 bases longer than the probe complement sequence. In some embodiments the extra length of the probe sequence compared to the probe complement sequence is as an overhang on one end of the probe sequence. In another embodiment, the probe sequence has an overhang of 6 or 7 bases over the probe complement sequence. In another embodiment, the probe sequence comprises 2′ O-methyl RNA. In another embodiment, the first coupling molecule comprises biotin. In another embodiment, the detectable marker comprises DIG. In another embodiment, the ID-tag sequence comprises an analog nucleotide such as L-DNA. In another embodiment, the ID-tag probe complex comprises a linker between the probe sequence and the ID-tag sequence.

In another aspect, an ID-tag detection complex is provided. The detection complex comprises a probe section. The probe section comprises a first ID-tag sequence connected to a probe sequence and the probe sequence is also connected to a detectable marker. The ID-tag detection complex further comprises a target segment. The target segment comprises a target sequence that is hybridized to the probe sequence. The ID-tag complex also comprises a detection segment that comprises a second ID-tag sequence that is hybridized to the first ID tag sequence. In one embodiment, the detection segment is located at a particular position in an array system.

In another aspect, a method of detecting a target segment in a sample is provided. The method comprises 1) contacting the ID-tag probe complex described above with a sample such that the probe sequence hybridizes to a target sequence in the sample, 2) contacting a second coupling molecule to the sample so that the second coupling molecule can bind to substantially all of the first coupling molecule, 3) removing substantially all of the second coupling molecule, and 4) detecting the detectable marker in the sample; thereby, detecting a target segment. In another embodiment the above method further comprises adding the remaining sample to an array, the array comprises a detection segment with a sequence that is complementary to the ID tag sequence of the ID tag probe complex at a first position, and detecting the presence of the detectable marker at the first position; thereby, detecting the presence the target segment in the sample. In one embodiment the target is RNA or miRNA. In one embodiment the first coupling molecule is biotin and the second coupling molecule is streptavidin. In one embodiment the array comprises multiple detection segments. In another embodiment the array comprises detection segments that are specific for the same or for different ID tag sequence.

In another embodiment the array comprises at least two different detection segments and sequences and there are at least two different ID tag probe complexes that are added to a sample. At least one of the ID tag probe complexes has a probe sequence that is different from a probe sequence in a different ID tag probe complex. In another embodiment at least three different ID tag sequences comprising three different ID-tag probes are used.

In another aspect, a method of detecting a target segment in a sample is provided. The method comprises contacting an ID-tag probe complex with a sample, removing substantially all first coupling molecule associated sequences from the sample, and detecting the presence of a detectable marker, thereby detecting a target segment in the sample.

In another aspect, an ID-tag complex kit is provided. The kit comprises an ID-tag probe complex. The ID-tag complex comprises 1) a probe section that comprises a detectable marker, an ID-tag sequence, and a probe sequence and 2) a probe complement section that comprises a first coupling molecule and a probe complement sequence. At least a portion of the probe complement sequence and the probe sequence are capable of hybridizing to each other. In one embodiment, the kit further comprises a second coupling molecule. In one embodiment, the kit further comprises an array. The array comprises a detection segment, having a second ID tag sequence that can hybridize to the first ID tag sequence. In one embodiment, the kit further comprises an RNase inhibitor or a means for isolating miRNA.

In another aspect, an indirect ID-tag complex is provided. The ID-tag complex comprises a probe section that comprises a probe sequence and a first coupling molecule. The probe sequence is connected to the first coupling molecule and the probe sequence can hybridize to a target sequence. The ID-tag complex further comprises a probe complement section that comprises an ID-tag sequence, a probe complement sequence, and a detectable marker. The probe complement sequence is connected to the ID-tag sequence and also connected to the detectable marker. The probe complement sequence is configured to hybridize and dissociate with at least a portion of the probe sequence. The probe sequence is at least one nucleotide longer than the probe complement sequence. In one embodiment, the detectable marker and the ID-tag sequences are on opposite ends of the probe complement sequence. In one embodiment, the probe sequence comprises an analog probe sequence. In one embodiment, the analog probe sequence comprises a nucleotide. In one embodiment, the analog probe sequence comprises a L-DNA. In one embodiment, the analog probe sequence comprises a RNA derivative. In one embodiment, the probe sequence will bind to RNA. In one embodiment, the probe sequence is complementary to a RNA target sequence. In one embodiment, the probe sequence will bind to miRNA. In a further embodiment, the probe sequence is complementary to a miRNA target sequence. In one embodiment, the probe sequence has an overhang compared to the probe complement sequence. In a further embodiment, the probe sequence has an overhang of 5-10 bases compared to the length of the probe complement sequence. In a further embodiment, the probe sequence has an overhang of 6 or 7 bases over the probe complement sequence. In one embodiment, the probe sequence comprises PNA or 2′ O-methyl RNA. In one embodiment, the first coupling molecule comprises biotin. In one embodiment, the detectable marker comprises DIG. In one embodiment, the ID-tag sequence comprises an analog nucleotide. In a further embodiment, the ID-tag sequence comprises L-DNA. In one embodiment, the ID-tag complex further comprises a linker between the probe complement sequence and the ID tag sequence.

In one aspect, an ID-tag detection complex is provided. The ID-tag detection complex comprises a probe complement section that comprises a first ID-tag sequence that is connected to a probe complement sequence. The probe complement sequence is also connected to a detectable marker. The ID-tag detection complex further comprises a detection segment that comprises a second ID tag sequence that is hybridized to the first ID tag sequence. In one embodiment, the detection segment is located at a particular position in an array system.

In one aspect, a method of detecting a target segment in a sample is provided. The method comprises contacting the ID-tag complex with a sample so that the probe sequence hybridizes to a target sequence in the sample, adding a second coupling molecule to the sample so that the second coupling molecule can bind to substantially all of the first coupling molecule, removing substantially all of the second coupling molecule and sequences associated therewith, and detecting a detectable marker, thereby detecting a target segment in a sample. In one embodiment, the method further comprises adding the remaining sample to an array. The array comprises a detection segment that is complementary to the ID-tag sequence of the ID-tag complex at a first position, and then detecting the presence of the detectable marker at the first position, thereby detecting the presence of the target segment in a sample. In one embodiment, the target is RNA or miRNA. In one embodiment, the first coupling molecule is biotin. In one embodiment, the second coupling molecule is streptavidin. In one embodiment, the array comprises multiple detection segments. In one embodiment, the detection segments are specific for a same probe complement sequence. In one embodiment, the detection segments are specific for a different probe complement sequences. In one embodiment, 1) the array comprises at least two different detection segments and 2) there are at least two different ID-tag complexes that are added to a sample. At least one of the ID-tag complexes has a probe sequence that is different from probe sequence in a different ID-tag complex. In one embodiment, the ID-tag sequence is different from another ID-tag sequence in the ID-tag complex. In one embodiment, at least three different ID-tag sequences that comprise three different ID tag probes are used.

In one aspect, an ID-tag complex kit is provided. The kit comprises 1) a probe complement section that comprises a first ID-tag sequence, a probe complement sequence, and a detectable marker, and 2) a probe section that comprises a first coupling molecule and a probe sequence. At least a portion of the probe sequence and the probe complement sequence are capable of hybridizing to each other. In one embodiment, the kit further comprises a second coupling molecule. In one embodiment, the kit further comprises an array; the array comprises a second ID-tag sequence. The second ID-tag sequence can hybridize to the first ID-tag sequence. In one embodiment, the kit further comprises an RNase inhibitor. In one embodiment, the kit further comprises a means for isolating miRNA. In one embodiment, the first coupling molecule is biotin and the second coupling molecule is streptavidin.

In another aspect, an ID-tag complex that comprises a hybridized set of sections with a means for binding to a target is provided. The binding to the target results in a dissociation of the complex. The dissociation provides a means for discriminating a dissociated target over an associated complex.

In another aspect, an ID-tag complex that comprises a set of sections is provided. The set of sections comprise a means for keeping a first and a second section together in the absence of a target sequence, a means for separating the two sections apart from each other in the presence of a target sequence, a means for distinguishing between a set of sections that are together and a set of sections that are apart from each other, and a means for identifying an identity of a first section.

In another aspect, an ID-tag complex is provided. The ID-tag complex comprises a probe section that comprises a probe sequence, an ID-tag sequence, and a detectable marker. The probe sequence is connected to the ID-tag sequence and the detectable marker. The ID-tag complex further comprises a probe complement section that comprises a probe complement sequence connected to a first coupling molecule. At least a portion of the probe sequence and the probe complement sequence are configured to hybridize to one another.

In another aspect, an ID-tag complex is provided. The ID-tag complex comprises a probe section that comprises 1) a probe sequence that comprises 2′ O-methyl RNA, 2) an ID-tag sequence that comprises L-DNA, and 3) DIG. The probe sequence is connected to the ID-tag sequence and DIG. The ID-tag complex further comprises a probe complement section that comprises 1) a probe complement sequence comprising DNA, and 2) biotin. The probe complement sequence is connected to the biotin. At least a portion of the probe sequence and the probe complement sequence are configured to hybridize to one another. The length of the probe sequence is at least 1 nucleotide greater than the length of the probe complement sequence. In one embodiment, there are a larger number of probe complement sections than there are probe sections. In a further embodiment, the number of probe complement sections outnumber the number of probe sections by a ratio of 2:1.

In one aspect, a method of detecting a target segment in a sample is provided. The method comprises contacting an ID-tag complex with a sample so that the probe sequence hybridizes to a target sequence in the sample. The ID-tag complex comprises a probe section that comprises 1) a probe sequence that comprises 2′ O-methyl RNA, 2) an ID-tag sequence comprising L-DNA, and 3) DIG. The probe sequence is connected to the ID-tag sequence and DIG. The ID-tag complex further comprises a probe complement section that comprises 1) a probe complement sequence that comprises DNA, and 2) biotin. The probe complement sequence is connected to the biotin. At least a portion of the probe sequence and the probe complement sequence are configured to hybridize to one another. The length of the probe sequence is at least 1 nucleotide greater than the length of the probe complement sequence. The method further comprises contacting a second coupling molecule to the sample so that the second coupling molecule can bind to substantially all of the first coupling molecule, removing substantially all of the second coupling molecule, detecting the detectable marker in the sample, thereby detecting a target segment, adding the remaining sample to an array. The array comprises an ID-tag detection sequence that is complementary to the ID-tag sequence of the ID-tag complex at a first position. The method further comprising detecting the presence of the detectable marker at the first position, thereby detecting the presence the target segment in the sample.

In another aspect, an ID-tag complex kit is provided. The kit comprises an ID-tag complex that comprises 1) a probe section comprising DIG, an ID-tag sequence comprising L-DNA, and a probe sequence comprising 2′ O-methyl RNA and 2) a probe complement section comprising a biotin attached to a probe complement sequence that comprises D-DNA. At least a portion of the probe complement sequence and said probe sequence are capable of hybridizing. The kit further comprises an ID-tag detection sequence that comprises L-DNA. The ID-tag detection sequence can bind to the ID-tag sequence. The kit further comprises an amount of streptavidin.

These and other features of the present teachings are set forth herein.

### BRIEF DESCRIPTION OF THE DRAWINGS

One of ordinary skill in the art will understand that the drawings, described below, are for illustration purposes only. The drawings are not intended to limit the scope of the present teachings in any way.

FIG. 1A is an illustration of one embodiment of an indirect ID-tag complex and a target sequence.

FIG. 1B is an illustration of one embodiment of the resulting distribution of the parts of an indirect ID-tag complex and a target sequence.

FIG. 1C is an illustration of one embodiment of a detection complex.

FIG. 2 is an illustration of one embodiment of an array of detection complexes from indirect ID-tag complexes.

FIG. 3 is a flow chart of one embodiment of a method of using an indirect ID-tag complex.

FIG. 4A is an illustration of one embodiment of a direct ID-tag complex and a target sequence.

FIG. 4B is an illustration of one embodiment of the resulting distribution of the parts of an indirect ID-tag complex and a target sequence.

FIG. 4C is an illustration of one embodiment of a detection complex.

FIG. 5 is an illustration of one embodiment of an array of detection complexes from direct ID-tag complexes.

FIG. 6 is a flow chart of one embodiment of a method of using a direct ID-tag complex.

FIG. 7 is a representation comparing the structure of DNA to PNA.

FIG. 8 is a representation comparing the structure of D-DNA to L-DNA.

### DESCRIPTION OF VARIOUS EMBODIMENTS

The present teaching is generally directed towards compositions and methods for identifying natural and analog nucleotide sequences. Generally, the compositions and methods involve a set of probes that are initially hybridized together in a duplex, also known as a “complex.” The presence of a target sequence results in the disruption of this duplex and the formation of a new duplex, resulting in the redistribution of the ID-tag complex's sequences and the properties of those sequences into the new complex. When used appropriately, this allows one to identify the presence or absence of particular target sequences in a sample. Additionally, some of the embodiments are especially useful for the identification of particularly short segments and/or sequences of nucleic acids that are traditionally difficult to detect and/or sequence. Additionally, by using the appropriate building blocks for each of the complex's sequences (e.g., PNA, L-DNA, RNA analogs) one can achieve additional favorable characteristics for the complex.

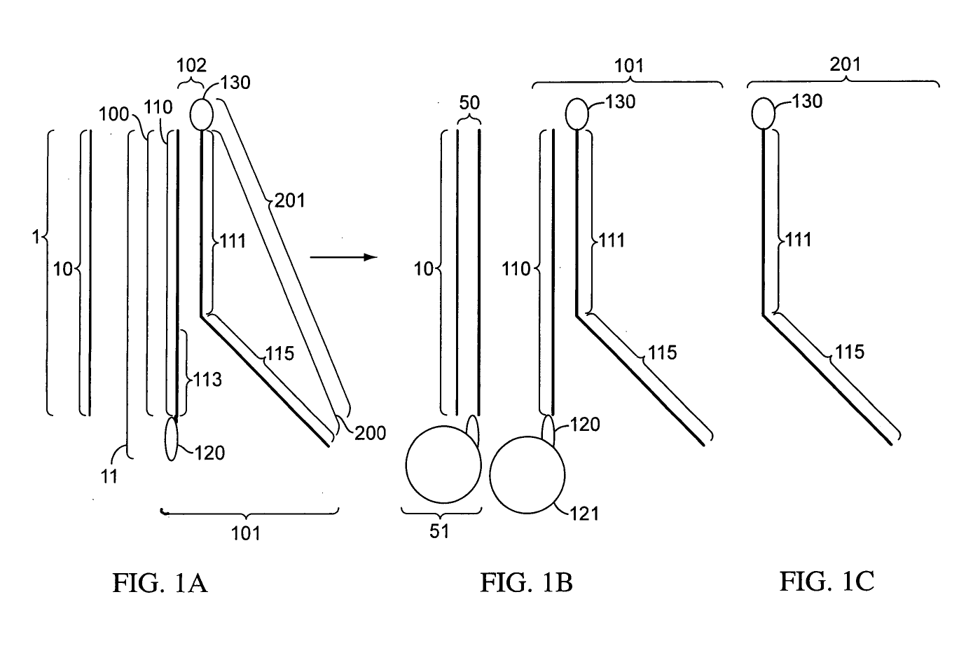

In one aspect, an “indirect ID-tag complex” is provided, which can be used to identify the presence or absence of a target sequence in a sample. With reference to FIG. 1A, the complex comprises two sections 11 and 201 that can comprise nucleic acids, analogs, or combinations thereof. The section 201 can comprise a detectable marker (DM) 130, a probe complement sequence 111, and an ID-tag sequence 115, that are connected to each other. The section 11 can comprise a probe sequence 110 and a first coupling molecule 120 that are connected to one another. The two sections 11 and 201 are initially bound to each other via the probe sequence 110 and the probe complement sequence 111, creating a duplex 102. However, the probe sequence 110 can dissociate e.g., break the duplex 102, to bind to a target sequence 10 to form a different, more stable, complex 51, see FIG. 1B. In some embodiments, the section 201 does not substantially bind to the target sequence 10 and/or other host sequence. The first coupling molecule 120 allows the section 11 to be separated from the other components, leaving section 201 as the remaining part of the initial ID-tag complex in a sample, see FIG. 1C. This remaining sample can be applied to an array 190, resulting in the formation of a detection complex 151, the observation of which indicates the presence of a target sequence. The “indirect” in the “indirect ID-tag complex” denotes that the section comprising the DM does not bind directly to the target sequence.

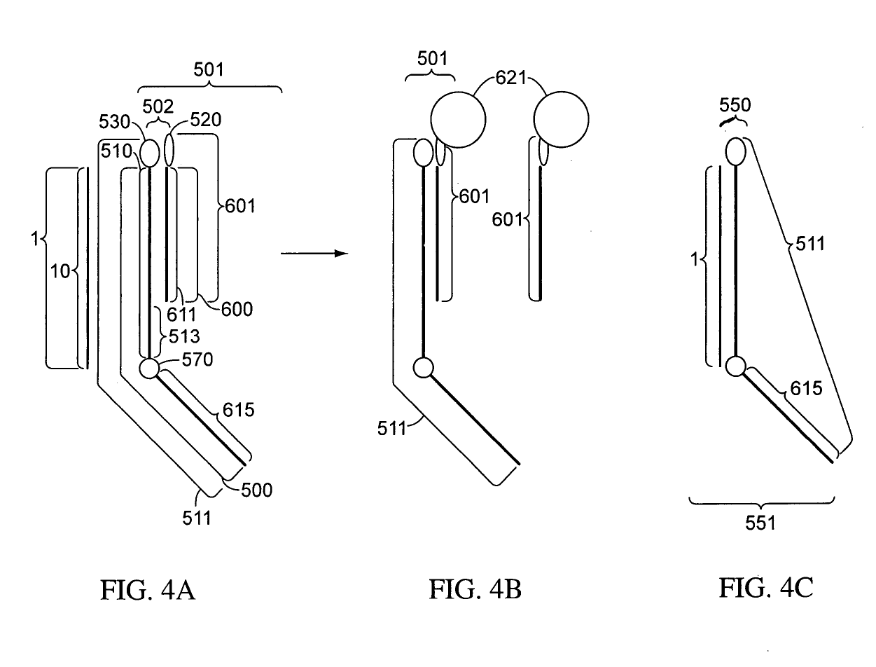

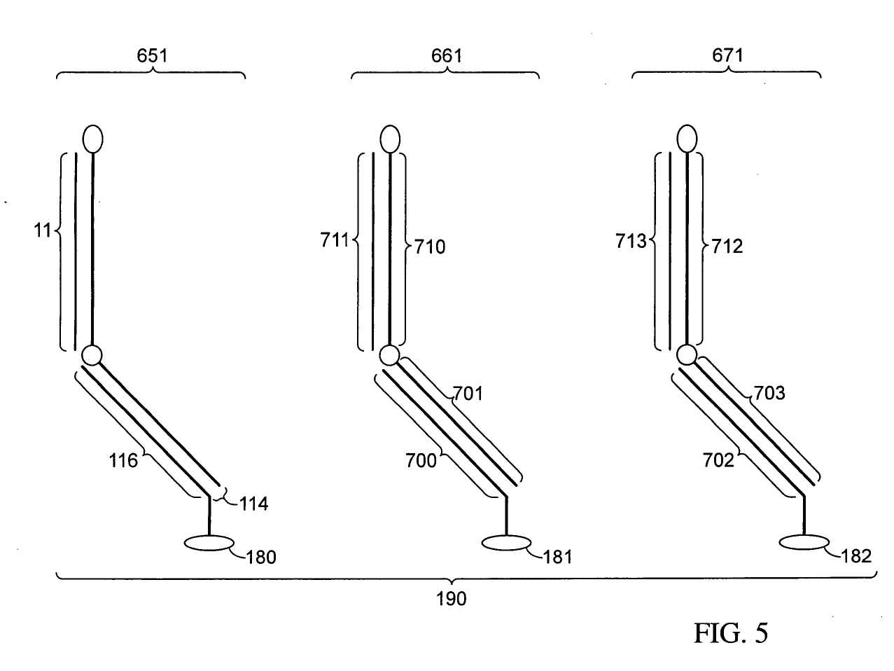

In another aspect, a “direct ID-tag complex” is provided, which can be used to identify the presence of a target sequence in a sample. Referring now to FIG. 4A, the complex 501 comprises two sections 511 and 601 that initially form a duplex 502 with one another. One of the sections 511 can dissociate the duplex 502 to bind to a target sequence 10 to form a different duplex 550. The other section 601 does not substantially bind to the target sequence 10. The section 511 that binds to the target sequence 10 comprises a detectable marker 530 and an ID-tag sequence 615. The section 601 comprises a first coupling molecule 520 that allows the section 601 to be removed from the sample, leaving section 511 as the remaining part of the initial ID-tag complex in a sample. This section 511-target 10 complex 551 can be applied to an array 190, resulting in the formation of a detection complex 651, the observation of which indicates the presence of a target sequence, FIG. 5. The “direct” in the “direct ID-tag complex” denotes that the section with the DM directly binds to the target sequence.

### DEFINITIONS

The term “configuration” refers to the spatial array of atoms that distinguishes stereoisomers (isomers of the same constitution) other than distinctions due to differences in conformation. Configurational isomers are stereoisomers that differ in configuration. Absolute configurations of the novel compositions described herein are defined by their particular chiral centers (e.g., sugar carbon atoms). The chiral carbons are designated by means of alphabetic symbols for rotation: R for rectus and S for sinister, defined by the bond priority rules of Cahn, Ingold, and Prelog (“Organic Chemistry”, Fifth Edition, J. McMurry, Brooks/Cole, Pacific Grove, Calif., pp. 315-319 (2000)), unless otherwise specified. In one embodiment, enantiomeric isomers of DNA are contemplated. In some embodiments, enantiomeric isomers of RNA are contemplated. In some embodiments, enantiomeric isomers of nucleotides and nucleobases are contemplated. Here, the configurational differences between the chiral carbons for normal DNA and analog DNA may be indicated by indicators such as “D-DNA” and “L-DNA,” which still refer to chirality of the molecule and which are defined further below.

The term “chimeric configurational” refers to a compound with covalently connected subunits comprising different stereochemical configurations.

“Nucleobase” means any nitrogen-containing heterocyclic moiety capable of forming Watson-Crick hydrogen bonds in pairing with a complementary nucleobase, including nucleobase analogs, e.g. a purine, a 7-deazapurine, or a pyrimidine. Typical nucleobases are the naturally occurring nucleobases adenine, guanine, cytosine, uracil, thymine, and analogs of the naturally occurring nucleobases (Seela, U.S. Pat. No. 5,446,139), e.g. 7-deazaadenine, 7-deazaguanine, 7-deaza-8-azaguanine, 7-deaza-8-azaadenine, inosine, nebularine, nitropyrrole (Bergstrom, J. Amer. Chem. Soc. 117:1201-09 (1995)), nitroindole, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine (Seela, U.S. Pat. No. 6,147,199), 7-deazaguanine (Seela, U.S. Pat. No. 5,990,303), 2-azapurine (Seela, WO 01/16149), 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, 4-methylindole, pyrazolo[3,4-D]pyrimidines, “PPG” (Meyer, U.S. Pat. Nos. 6,143,877 and 6,127,121; Gall, WO 01/38584), and ethenoadenine (Fasman, in Practical Handbook of Biochemistry and Molecular Biology, pp. 385-394, CRC Press, Boca Raton, Fla. (1989)). The term “nucleobase” includes those naturally occurring and those non-naturally occurring heterocyclic moieties commonly known to those who utilize nucleic acid technology or utilize peptide nucleic acid technology to generate polymers which can sequence-specifically bind to nucleic acids.

“Nucleoside” refers to a compound comprising a nucleobase linked to the C-1′ carbon of a sugar, such as ribose, arabinose, xylose, and pyranose, in the natural beta or the alpha anomeric configuration. The sugar can be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₁₂ alkyl, or C₃-C₁₄ aryl. Ribose examples include ribose, 2′-deoxyribose, 2′,3′-dideoxyribose, 2′-haloribose, 2′-fluororibose, 2′-chlororibose, and 2′-alkylribose, e.g. 2′-O-methyl, 4′-alpha-anomeric nucleotides, 1′-alpha-anomeric nucleotides (Asseline Nucl. Acids Res. 19:4067-74 (1991)), 2′-4′- and 3′-4′-linked and other “locked” or “LNA”, bicyclic sugar modifications (WO 98/22489; WO 98/39352; WO 99/14226). Exemplary LNA sugar analogs within a polynucleotide include the structures on page 4 of U.S. Patent Publication 2003/0198980, to Greenfield et al., on Oct. 23, 2003, where B is any nucleobase.

Sugars include modifications at the 2′- or 3′-position such as methoxy, ethoxy, allyloxy, isopropoxy, butoxy, isobutoxy, methoxyethyl, alkoxy, phenoxy, azido, amino, alkylamino, fluoro, chloro and bromo. Nucleosides and nucleotides include the natural D configurational isomer (D-form), as well as the L configurational isomer (L-form) (Beigelman, U.S. Pat. No. 6,251,666; Chu, U.S. Pat. No. 5,753,789; Shudo, EP0540742; Garbesi Nucl. Acids Res. 21:4159-65 (1993); Fujimori, J. Amer. Chem. Soc. 112:7435 (1990); Urata, Nucleic Acids Symposium Ser. No. 29:69-70 (1993)). When the nucleobase is purine, e.g. A or G, the ribose sugar is usually attached to the N⁹-position of the nucleobase. When the nucleobase is pyrimidine, e.g. C, T or U, the pentose sugar is usually attached to the N¹-position of the nucleobase (Kornberg and Baker, DNA Replication, 2^{nd} Ed., Freeman, San Francisco, Calif. (1992)).

“Nucleotide” refers to a phosphate ester of a nucleoside, as a monomer unit or within a nucleic acid. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g. .alpha.-thio-nucleotide 5′-triphosphates. For a review of nucleic acid chemistry, see: Shabarova, Z. and Bogdanov, A. Advanced Organic Chemistry of Nucleic Acids, VCH, New York, 1994.

The term “nucleic acid” refers to natural nucleic acids, artificial nucleic acids, analogs thereof, or combinations thereof.

As used herein, the terms “polynucleotide” and “oligonucleotide” are used interchangeably and mean single-stranded and double-stranded polymers of nucleotide monomers, including, but not limited to, 2′-deoxyribonucleotides (DNA) and ribonucleotides (RNA) linked by internucleotide phosphodiester bond linkages, e.g. 3′-5′ and 2′-5′, inverted linkages, e.g. 3′-3′ and 5′-5′, branched structures, or analog nucleic acids. Polynucleotides have associated counter ions, such as H⁺, NH₄⁺, trialkylammonium, Mg²⁺, Na⁺ and the like. A polynucleotide can be composed entirely of deoxyribonucleotides, entirely of ribonucleotides, or chimeric mixtures thereof. Polynucleotides can be comprised of nucleobase and sugar analogs. Polynucleotides typically range in size from a few monomeric units, e.g. 5-40 when they are more commonly frequently referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine, “C” denotes deoxycytidine, “G” denotes deoxyguanosine, and “T” denotes thymidine.

As used herein, the term “nucleobase sequence” is any section of a polymer which comprises nucleobase-containing subunits. Non-limiting examples of suitable polymers or polymer segments include oligonucleotides, oligoribonucleotides, peptide nucleic acids and analogs and chimeras thereof.

“Sequence” as compared to “segment.” While the terms may be used interchangeably in some circumstances, the term “segment” is generally meant to denote an entire physical piece of a nucleobase sequence, polynucleotide sequence, or combinations of both, although individual pieces or segments can be ligated together. A “sequence” is merely meant to denote those nucleobases, nucleotides, or both, that are required for a given function. Thus, a segment can have many sequences within it, meaning that it is one continuous chain with difference sequences with many possible functions. In comparison, a single sequence will normally only be one, or part of, a single segment. For example, in one embodiment, given a target sequence of CCATTACC, a probe segment with the sequence GGTAATGG, and a complementary probe sequence (i.e., probe complement), of TACC, the probe segment will comprise at least two sequences, one that can hybridize to the target (CCATTACC) and one that can hybridize to the complementary probe sequence (TACC), although probably not simultaneously for any substantial period of time. In general, a “section” will include all parts connected in a sufficiently stable manner, for example in some situations, a nonhybridized manner. Examples of sections include items 11 and 201 in FIG. 1. As will be appreciated by one of skill in the art, while a section can comprise more than just a segment and/or a sequence, it need not. Thus, a probe sequence that was not connected to anything, could be described as a probe section under the appropriate circumstances. Similarly, the probe segment in such a situation would also be the same as the probe sequence. An example of this is a target sequence that is the entire length of the target segment, which could also be the entirety of the target section.

An “analog” nucleic acid is a nucleic acid that is not normally found in a host to which it is being added or in a sample that is being tested. For example, the target sequence will not comprise an analog nucleic acid. This includes an artificial, synthetic, or combination thereof, nucleic acid. Thus, for example, in one embodiment, PNA is an analog nucleic acid, as is L-DNA and LNA (locked nucleic acids), iso-C/iso-G, L-RNA, O-methyl RNA, or other such nucleic acids. In one embodiment, any modified nucleic acid will be encompassed within the term analog nucleic acid. In another embodiment an analog nucleic acid can be a nucleic acid that will not substantially hybridize to native nucleic acids in a system, but will hybridize to other analog nucleic acids; thus, PNA would not be an analog nucleic acid, but L-DNA would be an analog nucleic acid. For example, while L-DNA can hybridize to PNA in an effective manner, L-DNA will not hybridize to D-DNA or D-RNA in a similar effective manner. Thus, nucleotides that can hybridize to a probe or target sequence but lack at least one natural nucleotide characteristic, such as susceptibility to degradation by nucleases or binding to D-DNA or D-RNA, are analog nucleotides in some embodiments. Of course, the analog nucleotide need not have every difference.

In some circumstances, not all of a segment needs to be of an analog nucleic acid in order for the segment to qualify as such. In one embodiment, only enough of the segment, sequence, or both is a nucleic acid analog so as to confer the desired properties of the nucleic acid analog onto the segment to which it is attached. Thus, for example, greater than 0% of each segment, sequence, or both will be of a nucleic acid analog. For example, minimal to 1, 1-2, 2-5, 5-10, 10-20, 20-40, 40-60, 60-80, or 80-100 percent of the sequence, segment, or both will be of an analog nucleic acid. In some embodiments, only nucleic acids that are immune from digestion from host nuclease enzymes will be considered analog nucleic acids. The analog nucleic acid, nucleotides, or both need not be restricted to DNA forms alone. As stated above PNA forms are included, as well as other forms of modified, e.g., artificial RNAs, for example, L-RNA, O-methyl RNA, LNA or other artificial RNAs. The bases comprising the analog nucleic acids need not be altered and can be able to bind with an effective level of specificity to the probe sequence. Phosphate ester analogs are encompassed within the term analog, and they include: (i) C₁-C₄ alkylphosphonate, e.g., methylphosphonate; (ii) phosphoramidate; (iii) C₁-C₆ alkyl-phosphotriester; (iv) phosphorothioate; and (v) phosphorodithioate.

As used herein, the terms “peptide nucleic acid” and “PNA” are any oligomer, linked polymer, or chimeric oligomer, comprising two or more PNA subunits (residues), including any of the compounds referred to, e.g., claimed as peptide nucleic acids in U.S. Pat. Nos. 5,539,082, 5,527,675, 5,623,049, 5,714,331, 5,736,336, 5,773,571 and 5,786,571 (all of which are hereby incorporated by reference). The term “Peptide Nucleic Acid” or “PNA” shall also apply to those nucleic acid mimics described in the following publications: Diderichsen et al., Tett. Lett. 37:475-478 (1996); Fujii et al., Bioorg. Med. Chem. Lett. 7:637-627 (1997); Jordan et al., Bioorg. Med. Chem. Lett. 7:687-690 (1997); Krotz et al., Tett. Lett. 36:6941-6944 (1995); Lagriffoul et al., Bioorg. Med. Chem. Lett. 4:1081-1082 (1994); Lowe et al., J. Chem. Soc. Perkin Trans. 1, (1997) 1:539-546; Lowe et al., J. Chem. Soc. Perkin Trans. 11:547-554 (1997); Lowe et al., J. Chem. Soc. Perkin Trans. 11:555-560 (1997); and Petersen et al., Bioorg. Med. Chem. Lett. 6:793-796 (1996).

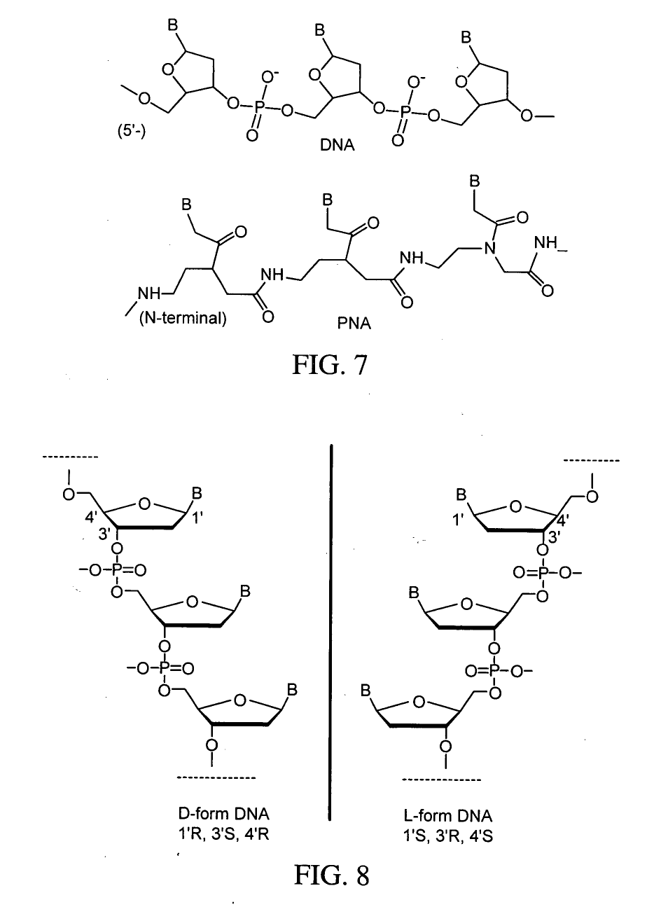

In one embodiment a PNA is a polymer comprising two or more PNA subunits of the formula 1 on page 6 of U.S. Patent Application 2003/0036059, to Coull et al., published Feb. 20, 2003. Each J is the same or different and is selected from the group consisting of H, R¹, OR¹, SR¹, NHR¹, NR¹₂, F, Cl, Br and I. Each K is the same or different and is selected from the group consisting of O, S, NH and NR¹. Each R¹ is the same or different and is an alkyl group having one to five carbon atoms which can optionally contain a heteroatom or a substituted or unsubstituted aryl group. Each A is selected from the group consisting of a single bond, a group of the formula; —(CJ₂)ₛ— and a group of the formula; —(CJ₂) ₅C(O)—, wherein, J is defined above and each s is an integer from one to five. The integer t is 1 or 2 and the integer u is 1 or 2. Each L is the same or different and is independently selected from the group consisting of J, adenine, cytosine, guanine, thymine, uridine, 5-methylcytosine, 2-aminopurine, 2-amino-6-chloropurine, 2,6-diaminopurine, hypoxanthine, pseudoisocytosine, 2-thiouracil, 2-thiothymidine, other naturally occurring nucleobase analogs, other non-naturally occurring nucleobases, substituted and unsubstituted aromatic moieties, biotin and fluorescein. In the most preferred embodiment, a PNA subunit consists of a naturally occurring or non-naturally occurring nucleobase attached to the aza nitrogen of the N-[2-(aminoethyl)]glycine backbone through a methylene carbonyl linkage. An example of a PNA polymer is shown in FIG. 7.

A L-DNA is a DNA whose three dimensional structure is different from D-DNA, the structure of D-DNA is shown in FIG. 8. In one embodiment, L-DNA comprises at least three structural differences as compared to D-DNA, as L-DNA is 1'S, 3'R, and 4'S. In another embodiment, L-DNA only has at least one difference as compared to D-DNA, for example 1'S, 3'R, 4'R; 1'R, 3'R, 4'S; or 1'R, 3R, 4'. In another embodiment, the enantiomeric differences are present within the bases themselves. The L-DNA need not be an exact mirrored structure of D-DNA in any respect apart from at least one enantiomeric bond difference. In this embodiment, it is only relevant that the “L-DNA” binds to the probe sequence, other similar nucleotide analog, or both, and does not bind the sequence type of the target. In another embodiment, while the target sequence can bind to a probe sequence, there is no significant species present that can bind to the L-DNA segment. This helps to make certain that signaling from the analog probe complex comes from the detection of a target sequence.

The term “chimeric configurational oligonucleotide” means a continuous oligonucleotide comprising nucleotides of different configurations. The term “chimeric configurational nucleic acid” means a continuous nucleic acid sequence comprising nucleic acids of different configurations. Chimeric configurational oligonucleotides can have one or more portions of L-form nucleotides and one or more portions of D-form nucleotides. The entire nucleotide need not be in the opposite conformation. The chimeric configurational oligonucleotide can comprise additional types of oligonucleotides as well.

“Self-indicating” analog probe complexes are probe complexes where the binding of the disruption of the duplex (102, 502 in FIGS. 1 & 4) because of the binding of part of the complex to the target sequence (10, in FIGS. 1 & 4)) results in an indication, (e.g., signal) occurring from the probe complex. As described below, the signal may originate from any part of the probe complex, e.g., an individual parts of the probe complex. This signal allows one to observe the presence of a target sequence in a sample. The signal e.g., indication, that occurs upon the detection of a sequence can be an increase in fluorescence of a donor fluorophore due to a decrease in a FRET interaction between a donor fluorophore on one section (11, or 201; 511, or 601 in FIGS. 1 & 4)) and the acceptor fluorophore on the complementary section (11, or 201; 511, or 601 in FIGS. 1 & 4)). Other such signaling events are discussed herein and are not limited to fluorescence.

“Polypeptide” refers to a polymer including proteins, synthetic peptides, antibodies, peptide analogs, and peptidomimetics in which the monomers are amino acids and are joined together through amide bonds. When the amino acids are alpha-amino acids, the L-optical isomer, the D-optical isomer, or both can be used. Additionally, unnatural amino acids, for example, valanine, phenylglycine and homoarginine are also included. Commonly encountered amino acids that are not gene-encoded can also be used. The amino acids used can be either the D- or L-optical isomer. In addition, other peptidomimetics can also be useful. For a general review, see Spatola, A. F., in Chemistry and Biochemistry of Amino Acids, Peptides and Proteins, B. Weinstein, eds., Marcel Dekker, New York, p. 267 (1983).

The term “amino acid” refers to naturally occurring and synthetic amino acids, as well as amino acid analogs that contain an amino group and a carboxylic acid group.

“Attachment site” refers to a site on a molecule, e.g. a quencher, a fluorescent dye, or a polynucleotide, to which is covalently attached, or capable of being covalently attached, another moiety. The attachment need only be sufficient for the use desired, and need not actually be covalent.

“Linker” refers to a chemical moiety in a molecule comprising a covalent bond or a chain of atoms that covalently attaches one molecule to another, e.g. a quencher to a polynucleotide. A “cleavable linker” is a linker that has one or more covalent bonds which can be broken by the result of a reaction, e.g., a condition. For example, an ester in a molecule is a linker that can be cleaved by a reagent, e.g. sodium hydroxide, resulting in a carboxylate-containing fragment and a hydroxyl-containing product.

“Reactive linking group” refers to a chemically reactive substituent or moiety, e.g., a nucleophile or electrophile, on a molecule that is capable of reacting with another molecule to form a covalent bond. Reactive linking groups include active esters, which are commonly used for coupling with amine groups. For example, N-hydroxysuccinimide (NHS) esters have selectivity toward aliphatic amines to form aliphatic amide products which are very stable. Their reaction rate with aromatic amines, alcohols, phenols (tyrosine), and histidine is relatively low. Reaction of NHS esters with amines under nonaqueous conditions is facile, so they are useful for derivatization of small peptides and other low molecular weight biomolecules. Virtually any molecule that contains a carboxylic acid or that can be chemically modified to contain a carboxylic acid can be converted into its NHS ester. NHS esters are available with sulfonate groups that have improved water solubility.

“Substituted” as used herein refers to a molecule wherein one or more hydrogen atoms are replaced with one or more non-hydrogen atoms, functional groups or moieties. For example, an unsubstituted nitrogen is —NH₂, while a substituted nitrogen is —NHCH₃. Exemplary substituents include but are not limited to halo, e.g., fluorine and chlorine, C₁-C₈ alkyl, sulfate, sulfonate, sulfone, amino, ammonium, amido, nitrile, nitro, alkoxy (—OR where R is C₁-C₁₂ alkyl), phenoxy, aromatic, phenyl, polycyclic aromatic, heterocycle, water-solubilizing group, and linking moiety.

“Alkyl” means a saturated or unsaturated, branched, straight-chain, branched, cyclic, or substituted hydrocarbon radical derived by the removal of one hydrogen atom from a single carbon atom of a parent alkane, alkene, or alkyne. Typical alkyl groups consist of 1-12 saturated and/or unsaturated carbons, including, but not limited to, methyl, ethyl, cyanoethyl, isopropyl, butyl, and the like.

“Alkyldiyl” means a saturated or unsaturated, branched, straight chain, cyclic, or substituted hydrocarbon radical of 1-12 carbon atoms, and having two monovalent radical centers derived by the removal of two hydrogen atoms from the same or two different carbon atoms of a parent alkane, alkene or alkyne. Typical alkyldiyl radicals include, but are not limited to, 1,2-ethyldiyl (—CH₂CH₂—), 1,3-propyldiyl (—CH₂CH₂CH₂—), 1,4-butyldiyl (—CH₂CH₂CH₂— CH₂—), and the like. “Alkoxydiyl” means an alkoxyl group having two monovalent radical centers derived by the removal of a hydrogen atom from the oxygen and a second radical derived by the removal of a hydrogen atom from a carbon atom. Typical alkoxydiyl radicals include, but are not limited to, methoxydiyl (—OCH₂—) and 1,2-ethoxydiyl or ethyleneoxy (—OCH₂CH₂—). “Alkylaminodiyl” means an alkylamino group having two monovalent radical centers derived by the removal of a hydrogen atom from the nitrogen and a second radical derived by the removal of a hydrogen atom from a carbon atom. Typical alkylaminodiyl radicals include, but are not limited to —NHCH₂—, —NHCH₂CH₂—, and —NHCH₂CH₂CH₂—. “Alkylamidediyl” means an alkylamide group having two monovalent radical centers derived by the removal of a hydrogen atom from the nitrogen and a second radical derived by the removal of a hydrogen atom from a carbon atom. Typical alkylamidediyl radicals include, but are not limited to —NHC(O)CH₂—, —NHC(O)CH₂CH₂—, and —NHC(O)CH₂CH₂CH₂—.

“Aryl” means a monovalent aromatic hydrocarbon radical of 5-14 carbon atoms derived by the removal of one hydrogen atom from a single carbon atom of a parent aromatic ring system. Typical aryl groups include, but are not limited to, radicals derived from benzene, substituted benzene, naphthalene, anthracene, biphenyl, and the like, including substituted aryl groups.

“Aryldiyl” means an unsaturated cyclic or polycyclic hydrocarbon radical of 5-14 carbon atoms having a conjugated resonance electron system and at least two monovalent radical centers derived by the removal of two hydrogen atoms from two different carbon atoms of a parent aryl compound, including substituted aryldiyl groups.

“Substituted alkyl”, “substituted alkyldiyl”, “substituted aryl” and “substituted aryldiyl” mean alkyl, alkyldiyl, aryl and aryldiyl respectively, in which one or more hydrogen atoms are each independently replaced with another substituent. Typical substituents include, but are not limited to, F, Cl, Br, I, R, OH, —OR, —SR, SH, NH₂, NHR, NR₂, —⁺NR₃, —N—NR₂, —CX₃, —CN, —OCN, —SCN, —NCO, —NCS, —NO, —NO₂, —N₂⁺, —N₃, —NHC(O)R, —C(O)R, —C(O)NR₂—S(O)₂O⁻, —S(O)₂R, —OS(O)₂OR, —S(O)₂NR, —S(O)R, —OP(O)(OR)₂, —P(O)(OR)₂, —P(O)(O—)₂, —P(O)(OH)₂, —C(O)R, —C(O)X, —C(S)R, —C(O)OR, —CO₂⁻, —C(S)OR, —C(O)SR, —C(S)SR, —C(O)NR₂, —C(S)NR₂, —C(NR)NR₂, where each R is independently —H, C₁-C₆ alkyl, C₅-C₁₄ aryl, heterocycle, or linking group. Substituents also include divalent, bridging functionality, such as diazo (—N—N—), ester, ether, ketone, phosphate, alkyldiyl, and aryldiyl groups.

“Heterocycle” refers to a molecule with a ring system in which one or more ring atoms is a heteroatom, e.g. nitrogen, oxygen, and sulfur (as opposed to carbon).

“Enzymatically extendable” refers to a nucleotide which is: (i) capable of being enzymatically incorporated onto a terminus of a polynucleotide through the action of a polymerase enzyme, and (ii) capable of supporting further primer extension. Enzymatically extendable nucleotides include nucleotide 5′-triphosphates, i.e. dNTP and NTP, and labelled forms thereof.

“Enzymatically incorporatable” refers to a nucleotide which is capable of being enzymatically incorporated onto a terminus of a polynucleotide through the action of a polymerase enzyme. Enzymatically incorporatable nucleotides include dNTP, NTP, and 2′,3′-dideoxynucleotide 5′-triphosphates, i.e. ddNTP, and labelled forms thereof.

“Terminator nucleotide” means a nucleotide which is capable of being enzymatically incorporated onto a terminus of a polynucleotide through the action of a polymerase enzyme, but cannot be further extended, i.e. a terminator nucleotide is enzymatically incorporatable, but not enzymatically extendable. Examples of terminator nucleotides include ddNTP and 2′-deoxy, 3′-fluoro nucleotide 5′-triphosphates, and labelled forms thereof.

“Primer” means an oligonucleotide of defined sequence that is designed to hybridize with a complementary, primer-specific portion of a target sequence, a probe, a ligation product, or any combination of the foregoing, and undergo primer extension. A primer functions as the starting point for the polymerization of nucleotides (Concise Dictionary of Biomedicine and Molecular Biology, CPL Scientific Publishing Services, CRC Press, Newbury, UK (1996)).

The term “duplex” means an intermolecular or intramolecular double-stranded portion of a nucleic acid that is base-paired through Watson-Crick, Hoogsteen, or other sequence-specific interactions of nucleobases. As examples, a duplex can consist of a primer and a template strand, or a probe and a target strand. A “hybrid” means a duplex, triplex, or other base-paired complex of nucleic acids interacting by base-specific interactions, e.g. hydrogen bonds.

The term “primer extension” means the process of elongating a primer that is annealed to a target in the 5′ to 3′ direction using a template-dependent polymerase. According to certain embodiments, with appropriate buffers, salts, pH, temperature, and nucleotide triphosphates, including analogs and derivatives thereof, a template dependent polymerase incorporates nucleotides complementary to the template strand starting at the 3′-end of an annealed primer, to generate a complementary strand.

The term “label” refers to any moiety that can be associated with a polynucleotide and: (i) provide a detectable signal; (ii) interact with a second label to modify the detectable signal provided by the second label, e.g. FRET; (iii) stabilizes hybridization, e.g., duplex formation; (iv) confers a capture function, e.g., hydrophobic affinity, antibody/antigen, ionic complexation, (v) change a physical property, such as electrophoretic mobility, hydrophobicity, hydrophilicity, solubility, or chromatographic behavior, or (vi) any combination of the foregoing.

“Detectable marker”, “detection markers” “DM,” “detection moieties,” “label,” or other similar terms are used interchangeably. Detectable markers need not be visible through emission based methods. Thus, in one embodiment, a detectable marker is one that is detectable through more traditional means, such as antibody binding assays to the detectable marker. In other embodiments, the excitation, emission, and/or adsorption spectra of the detectable marker can be used for observation. Similarly, the marker modifier can be used to create changes in any of those spectra. In another embodiment, the detectable marker can create and/or inhibit some product that is itself detectable. In embodiments where the detectability of the detectable marker is modifiable to indicate the presence and/or absence of an ID-tag complex and/or detection complex (which in turn indicates the absence or presence of the target sequence), the detectable marker can still be modifiable in terms of its detection, although this can derive from something other than a marker modifier. The detectable marker need not be covalently associated with the segments. In one embodiment, the absorption properties of the marker can be changed; thus, the detection is not of something emitted, but of something absorbed by the marker.

In one embodiment, the detectable marker emits a form of light and/or is sensitive to magnetic fields. The detectable marker can be a superparamagnetic nanoparticle or similar MRI detectable particle. The detectable marker can also be a fluorescent moiety. A fluorescent moiety is a moiety that fluoresces light, although the light need not be of the “visible” wavelength. A fluorescent marker is a compound that specifically emits light that can be detected. A fluorescent quencher is a label that alters the fluorescence of a fluorescent marker.

Marker modifiers can be used to further allow the detection of a target sequence. “Marker modifiers,” “MM” or other similar terms are compounds that allow for the detection of whether or not the sections of a complex have dissociated from each other, indicating the presence of a target sequence. The dissociation need not be complete. Anything that allows the observation of the probe sequence binding to a target sequence can be a marker modifier. Marker modifiers (MMs) can be paired with DMs so that a first signal is generated by the DM; when the marker modifier and the DM are separated, a second signal is generated by the DM. Events which result in the pairing and separation of DMs and marker modifiers can thus be observed through changes in these signals. Thus, Beta-field shielders, when paired with a superparamagnetic DM, can be considered marker modifiers. Fluorescent modifiers, e.g., quenchers that alter the fluorescence characteristics of a fluorescent marker can also be a modifier. The marker modifier can also be a fluorescent probe that alters the fluorescence of the marker itself. The fluorescence modification need not be FRET based. The marker modifiers need not directly modify an emitted signal from a detectable marker.

As used herein, “energy transfer” refers to the process by which the excited state energy of an excited group, e.g. fluorescent reporter dye, is conveyed through space and/or through bonds to another group, e.g. a quencher moiety, which can attenuate (quench), otherwise dissipate, or transfer the energy. Energy transfer can occur through fluorescence resonance energy transfer, direct energy transfer, and other mechanisms, such as changes in the local environment of a marker (label) or changes in the mobility of the marker (label) itself. The exact energy transfer mechanisms is not limiting to the present embodiments. It is to be understood that any reference herein to energy transfer encompasses all of these mechanistically-distinct phenomena.

“Energy transfer pair” refers to any two moieties that participate in energy transfer. Typically, one of the moieties acts as a fluorescent reporter, i.e. donor, and the other acts as a fluorescence quencher, i.e. acceptor (“Fluorescence resonance energy transfer.” Selvin P. (1995) Methods Enzymol 246:300-334; dos Remedios C. G. (1995) J. Struct. Biol. 115:175-185; “Resonance energy transfer: methods and applications.” Wu P. and Brand L. (1994) Anal Biochem 218:1-13). Fluorescence resonance energy transfer (FRET) is a distance-dependent interaction between two moieties in which excitation energy, i.e. light, is transferred from a donor (“reporter”) to an acceptor without emission of a photon. The acceptor can be fluorescent and emit the transferred energy at a longer wavelength, or it can be non-fluorescent and serve to diminish the detectable fluorescence of the reporter (quenching). FRET can be either an intermolecular or intramolecular event, and is dependent on the inverse sixth power of the separation of the donor and acceptor, making it useful over distances comparable with the dimensions of biological macromolecules. Thus, the spectral properties of the energy transfer pair as a whole change in some measurable way if the distance between the moieties is altered by some detectable amount. Self-quenching probes incorporating fluorescent donor-nonfluorescent acceptor combinations have been developed primarily for detection of proteolysis (Matayoshi, (1990) Science 247:954-958) and nucleic acid hybridization (“Detection of Energy Transfer and Fluorescence Quenching” Morrison, L., in Nonisotopic DNA Probe Techniques, L. Kricka, Ed., Academic Press, San Diego, (1992) pp. 311-352; Tyagi S. (1998) Nat. Biotechnol. 16:49-53; Tyagi S. (1996) Nat. Biotechnol 14:303-308). In most applications, the donor and acceptor dyes are different, in which case FRET can be detected by the appearance of sensitized fluorescence of the acceptor or by quenching of donor fluorescence.

The term “quenching” refers to a decrease in signal detectable moiety caused by a quencher moiety, regardless of the mechanism. For example, illumination of a fluorescent marker in the presence of a quencher leads to an emission signal that is less intense than expected, or even completely absent. The quencher can block 0-100% of the signal. For example, a quencher blocks 0-1, 1-10, 10-20, 20-30, 30-40, 40-50, 50-60, 60-70, 70-80, 80-90, 90-95, 95-99, or 99-99.95, 99.95-99.98, 99.98-99.99, 99.99-100 percent of the signal of the detectable marker. In FRET based systems, quenching has its normal connotations. Shifts in fluorescent emission will also be considered as an adequate means for observing the changes in fluorescence from a detectable marker or the marker modifier. Thus, while the DM or the MM can emit just as much light, the emission of that light can be at different wavelengths than it was when the two segments were hybridized or separated. Thus, the change in signal between the hybridized analog probe complex and the separated analog probe complex need not be an absolute change in fluorescence intensity, since any change, e.g. absorption, emission spectra, intensity, that can be correlated to the two states of the probe can be sufficient for certain embodiments to function as desired.

The term “first coupling molecule” (“CM1”) refers to a molecule that can bind, be bound to another molecule, or both, with sufficient strength so that the first coupling molecule and an appropriately associated molecule with the first coupling molecule (e.g., a probe complement segment 600 or probe segment 100 as in FIGS. 1A and 4A) can be removed from a solution. In some embodiments, the first coupling molecule can allow the complete removal from solution of all molecules covalently associated with it. In other embodiments, the first coupling molecule, when paired with an appropriate second coupling molecule, allows for an effective amount of the molecule associated with the first coupling molecule to be removed from a sample via the removal of the second coupling molecule. For example, removal of 100 to 1% or less, for example, 100-99, 99-95, 95-90, 90-80, 80-70, 70-50, 50-30, 30-20, 20-10, 10-1 percent or less of the first coupling molecule, and thus, the molecule associated with the first coupling molecule, can be sufficient to allow the compositions and methods disclosed herein to perform as desired. The required amount will be determined according to the teachings herein and the knowledge of one of ordinary skill in the art, as appropriate for a particular situation. Any molecule with the desired particular characteristics can be useful as a first coupling molecule. The first coupling molecule should not interfere with the other functions of the probe. The first coupling molecule can bind sufficiently tightly and with a sufficiently long duration so as to allow the first coupling molecule to bind to, or be bound by, the second coupling molecule and for both to be removed from the sample, as well as any molecule associated with the first coupling molecule. As will be appreciated by one of skill in the art, the molecules associated with the first coupling molecule can vary depending upon the embodiment. Additionally, the first coupling molecule need not be covalently attached to the associated molecule, as long as the interaction between the first coupling molecule and the associated molecule is sufficiently stable so as to allow removal of the associated molecule from the sample, through the use of the first coupling molecule. Examples of such a molecule include biotin, avidin, streptavidin, epitopes and paratopes from antigens and antibodies.

The term “second coupling molecule” (“CM2”) refers to the molecule that is capable of binding to the first coupling molecule so that the first coupling molecule can function as described above. The actual act of binding of the first coupling molecule to the second coupling molecule, or of the second coupling molecule to the first coupling molecule is not important to the functionality of the embodiments and need not be limited by the terms used. In other words, the first coupling molecule can be the molecule that binds to the CM2, or the CM2 can be the molecule that binds to the CM1. Alternatively, they both can bind to each other. Examples of such molecules include biotin, avidin, streptavidin, epitopes and paratopes such as from antigens and antibodies. Of course, as will be appreciated by one of skill in the art in light of the present disclosure and unless otherwise specified herein, the precise placement of a CM1 or CM2 on any one particular probe complement segment or probe segment is freely interchangeable. In other words, either one of the CM1 or CM2 can be on either one of the segments to which they are to be attached, as long as they allow for the removal of the sequence associated with the CM1 or the CM2. The figures included herein are for representation purposes only and are not meant to denote limitations on the claims.

The term “probe” sequence is meant to denote a sequence of nucleic acids, naturally occurring or an analog thereof, such as PNA or various forms of DNA or RNA, which can bind to a target sequence with some sufficient degree of specificity and relative stability. Various combinations of nucleic acid types may also be employed. The probe sequence binds to the target sequence through base pairing of the probe sequence, contained within the probe segment to the target sequence, contained within the target sequence. In one embodiment, the probe sequence is made of PNA. In another embodiment, the probe sequence comprises 2′ O-methyl RNA or other analogs of RNA such as 2′ O-fluoro or 2′ O-ethyl. The type of material that the probe is made out of can bind the target sequence (e.g., RNA) more tightly than it binds the probe complement sequence (e.g., DNA).

The term “probe complement,” “PC,” and “complementary probe” sequence are meant to denote a sequence that is complementary to a sequence of a probe sequence. The probe complement sequence can hybridize to the probe sequence and/or segment but is not the target sequence. Additionally, the binding properties of the probe complement and the probe are generally different compared to that of the probe and the target. The probe complement need not bind, and preferably does not bind, to the entire probe sequence. The probe complement can be displaced from the ID tag coupler by the binding of a part of the probe sequence with a part of the target sequence. The probe-target sequence duplex can be more stable than the probe-probe complement hybridization. This amount of increased stabilization can be any amount; for example, 1-5, 5-10, 10-20, 20-30, 30-40, 40-60, 60-80, 80-100, 100-151, 151-200, 200-300, 300-500, percent more stable or more, as determined through means known to one of skill in the art. The probe complement sequence can be 100% complementary to the relevant portion of the probe sequence. However, this number can be less, as long as they are substantially similar so that dissociation occurs significantly through binding of the probe sequence to the target sequence. As will be appreciated by one of skill in the art, the terms probe, target, probe complement, ID-tag, etc., can be used to identify particular sequences, segments, sections, and complexes, assuming that they are contained with the particular sequence, segment, section, and complex. In some embodiments, the probe complement is configured so as to not interfere with the association of the probe and the target and so as to allow the rapid and efficient dissociation of the probe-probe complement duplex. One manner in which the probe complement can be so configured is to use an analog nucleic acid, such as L-DNA, when the target sequence is not a complementary analog sequence. The probe complement sequence can be comprised of any nucleic acid, nucleic acid analog, or combination thereof, with the requirements from the teachings herein and the particular target sequence to be detected. For example, it can be made from L-DNA, L-RNA, PNA, D-DNA, etc.

“Target,” “target polynucleotide,” “target sequence,” or similar term means a specific polynucleotide sequence, the presence or absence of which is to be detected. The sequence can be the subject of hybridization with a complementary polynucleotide, e.g. a primer or probe. The target sequence can be composed of DNA, RNA, analogs thereof, and including combinations thereof. The target can be single-stranded or double-stranded. In primer extension processes, the target polynucleotide which forms a hybridization duplex with the primer can also be referred to as a “template.” A template serves as a pattern for the synthesis of another, complementary nucleic acid (Concise Dictionary of Biomedicine and Molecular Biology, CPL Scientific Publishing Services, CRC Press, Newbury, UK (1996)). A target sequence can be derived from any living, or once living, organism, including but not limited to prokaryote, eukaryote, plant, animal, and virus. The target sequence can originate from a nucleus of a cell, e.g., genomic DNA, or can be extranuclear nucleic acid, e.g., plasmid, mitrochondrial nucleic acid, various RNAs, and the like. The target nucleic acid sequence can be first reverse-transcribed into cDNA if the target nucleic acid is RNA, if so desired. A variety of methods are available for obtaining a target nucleic acid sequence for use with the compositions and methods described herein. When the target sequence is obtained through isolation from a biological sample, possible isolation techniques include (1) organic extraction followed by ethanol precipitation, e.g., using a phenol/chloroform organic reagent (e.g., Ausubel et al., eds., Current Protocols in Molecular Biology Volume 1, Chapter 2, Section I, John Wiley & Sons, New York (1993)), or an automated DNA extractor (e.g., Model 341 DNA Extractor, Applied Biosystems, Foster City, Calif.); (2) stationary phase adsorption methods (e.g., Boom et al., U.S. Pat. No. 5,234,809; Walsh et al., Biotechniques 10(4): 506-513 (1991)); and (3) salt-induced DNA precipitation methods (e.g., Miller et al., Nucleic Acids Research, 16(3): 9-10 (1988)). In one embodiment, the target sequence can be mRNA. In another embodiment the term “target sequence” can be any sequence of nucleobases in a polymer which is sought to be detected. The “target sequence” can comprise the entire polymer or can be a subsequence of the nucleobase polymer that is unique to the polymer of interest. Without limitation, the polymer comprising the “target sequence” can be a nucleic acid, a peptide nucleic acid, a chimera, a linked polymer, a conjugate or any other polymer comprising substituents (e.g. nucleobases) to which the PNA probe sequence can bind in a sequence specific manner. The target sequence can include any nature of nucleotide as well, for example, PNA, cDNA, mRNA, antisense RNA, siRNA, or microRNA (for a discussion of miRNA see Grishok et al., Cell, 106:2334 (2001); Carrington and Ambros, Science 301:336-338 (2003)). As will be appreciated by one of skill in the art, there can be a difference between a target sequence and a “sequence to be detected.” Generally, in the discussion herein, the target sequence is the sequence that can hybridize to the probe sequence. However, as will be appreciated by one of skill in the art, this sequence need not be the sequence that one is particularly interested in, as the sequence of interest, e.g., sequence to be detected may be located elsewhere on a segment that also contains the target sequence. However, if the two can be correlated in such a fashion, then the detection can be similarly correlated.

An “identification tag,” “identifying tag,” “ID-tag,” “ID” sequence, or similar term can describe the segment, section, and sequence that is capable of being used to identify that particular segment or sequence. A set of hybridized identifying tags involves two sequences, which can hybridize to each other to an extent that will allow the effective detection of the hybridization. This hybridization of the two parts of the identification tag hybrid can also be specific enough to allow one to distinguish between the presence and absence of particular identifying tag sequences. In one embodiment, one identifying tag is attached to a target to be detected while another identifying tag is attached to a substrate at a known position, location, or in a determinable location. Since the two identification tag sequences can hybridize together, the presence of the target in the system can be detected by looking for hybridization of the identifying tag. Alternative embodiments are discussed in greater detail below. In some embodiments, this can also be described as a zip-coded tag or sequence.

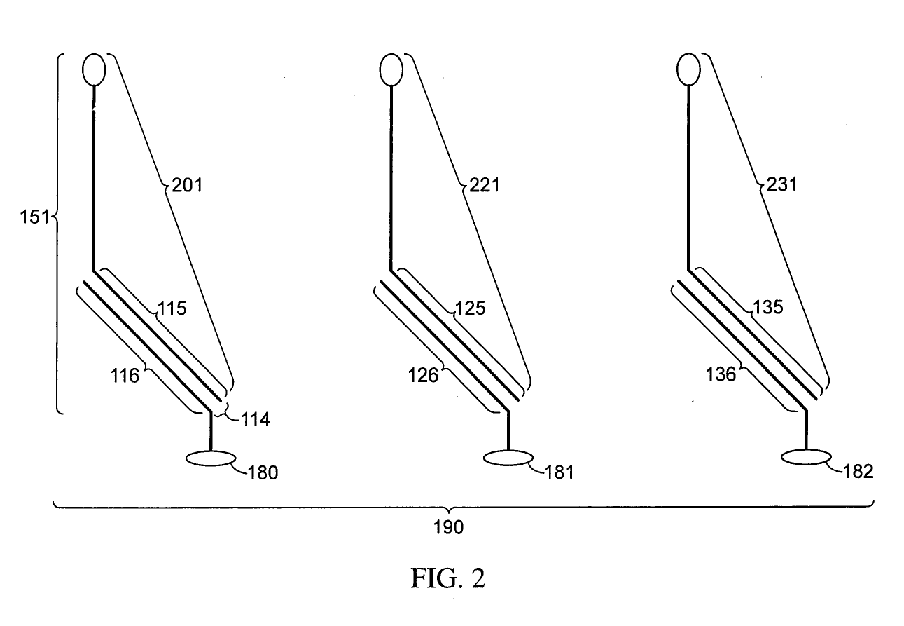

An “ID tag” sequence will generally refer to a sequence of the hybridized identification tag. A “set” or “pair” of ID-tags will generally denote two ID-tags that can hybridize together. A hybridized or duplexed ID tag refers to two ID-tags that are hybridized together. A “detection” ID-tag refers to the ID-tag that is in the known or knowable detection format, for example, an ID-tag that is part of an array (e.g., 116, 700, and 702 in FIGS. 2 & 5). A “target” or “inquiry” ID-tag refers to the ID-tag whose presence or absence can indicate the presence, absence, concentration or other information concerning a target sequence (e.g., 115 and 615 in FIGS. 1, 2, 4, & 5). Identifying portion sequences and identifying portion complement sequences can be selected by any suitable method, for example, but not limited to, computer algorithms such as described in PCT Publication Nos. WO 96/12014 and WO 96/41011 and in European Publication No. EP 799,897; and the algorithm and parameters of SantaLucia (Proc. Natl. Acad. Sci. 95:1460-65 (1998)). Descriptions of identifying portions can be found in, among other places, U.S. Pat. No. 6,309,829 (referred to as “tag segment” therein); U.S. Pat. No. 6,451,525 (referred to as “tag segment” therein); U.S. Pat. No. 6,309,829 (referred to as “tag segment” therein); U.S. Pat. No. 5,981,176 (referred to as “grid oligonucleotides” therein); U.S. Pat. No. 5,935,793 (referred to as “identifier tags” therein); and PCT Publication No. WO 01/92579 (referred to as “addressable support-specific sequences” therein). As will be appreciated by one of skill in the art, the ID-tag sequence can be made of practically any nucleic acid, nucleic acid analog, or mixture thereof,as long as the desired functionality is maintained. For example, the ID-tag sequence or sequences can comprise L-DNA or L-RNA, as well as other forms of nucleic acids. The set of ID-tag sequences can also be created so that each set is unique and sets are unlikely to form duplexes out of the set. Additionally, they can be selected so as to reduce or eliminate the risk that sequences in the sample will bind to the ID-tag sequence.

The term “complex” generally denotes the composition formed between two sections when a duplex is formed between the two sections. (e.g., 101, 51, 151, 501, 551, and 651).

The terms “annealing” and “hybridizing” are used interchangeably and mean the base-pairing interaction of one nucleic acid with another nucleic acid that results in formation of a duplex or other higher-ordered structure. The primary interaction is base specific, i.e. A/T and G/C, by Watson/Crick and Hoogsteen-type hydrogen bonding.

The term “solid support” refers to any solid phase material upon which an oligonucleotide is synthesized, attached or immobilized. Solid support encompasses terms such as “resin”, “solid phase”, and “support”. A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as, for example, glass, silica, controlled-pore-glass (CPG), or reverse-phase silica. The configuration of a solid support can be in the form of beads, spheres, particles, granules, a gel, a surface, or combinations thereof. Surfaces can be planar, substantially planar, or non-planar. Solid supports can be porous or non-porous, and can have swelling or non-swelling characteristics. A solid support can be configured in the form of a well, depression or other container, vessel, feature or location or position. A plurality of solid supports can be configured in an array at various locations, e.g., positions, addressable for robotic delivery of reagents, or by detection means including scanning by laser illumination and confocal or deflective light gathering.

“Array” or “microarray” encompasses an arrangement of polynucleotides present on a solid support or in an arrangement of vessels. Certain array formats are referred to as a “chip” or “biochip” (M. Schena, Ed. Microarray Biochip Technology, BioTechnique Books, Eaton Publishing, Natick, Mass. (2000)). An array can comprise a low-density number of addressable locations, e.g. 1 to about 12, medium-density, e.g. about a hundred or more locations, or a high-density number, e.g. a thousand or more. Typically, the array format is a geometrically-regular shape which allows for fabrication, handling, placement, stacking, reagent introduction, detection, and storage. The array can be configured in a row and column format, with regular spacing between each location. Alternatively, the locations can be bundled, mixed, or homogeneously blended for equalized treatment and/or sampling. An array can comprise a plurality of addressable locations configured so that each location is spatially addressable for high-throughput handling, robotic delivery, masking, and/or sampling of reagents and/or by detection means including scanning by laser illumination and confocal and/or deflective light gathering. The array may comprise one or more “addressable locations,” e.g., “addressable positions,” that is, physical locations that comprise a known type of molecule. In one embodiment an addressable location comprises more than one type of ID-tag sequence. However, the types of ID-tag sequence present at each location are known or can be determined.

A “suspension array” is one alternative composition or method for performing analyte detection and/or quantification. In a suspension array, the solid phase consists of particles in solution. Each particle member of the array has a characteristic, such as a shape, pattern, chromophore or fluorophore that uniquely identifies the particle, e.g., bead. Each uniquely identified particle member has a unique ID-tag sequence attached to its surface. In a two-dimensional array, the identity of the ID-tag sequence can be determined by location in the two-dimensional surface. In a suspension array, the identity of the ID-tag sequence can be determined by the unique characteristic of the particle member.

Typically, the identity of any targets present is of interest. Thus, it is necessary to identify the type of ID-tag sequence on each bead at each location in the array so that the binding of different targets can be distinguished. This may be achieved by individually placing beads with known ID-tag sequences in the array. Alternatively, the beads may be randomly distributed in the array and the specific location of individual beads in the array determined after the array is formed. This may be accomplished by any method known in the art. For example, a fluorescently labeled oligonucleotide that is complementary to a particular ID-tag sequence may be used to determine the exact location of beads that comprise that ID-tag sequence.

In one embodiment, an array is used to detect the presence of two or more target sequences in a sample. ID-tag-coupled beads are prepared that are specific for each target sequence to be detected. The different types of ID-tag-coupled beads are then placed into solution in separate vessels, so that each vessel contains only beads comprising ID-tag sequences that are specific for a particular target sequence. Preferably, the ID-tag coupled beads are placed in solution in the wells of a microtiter plate. The location of the wells comprising specific types of ID-tag coupled beads is noted. A particular sample of interest is then divided between each of the wells comprising a specific type of ID-tag coupled beads. The beads are then washed to remove the sample and the detectable marker can be measured. The identity of target sequences that are present in the sample can then be determined by comparing direct results, e.g., a change in fluorescence, to the noted location of the particular types of ID-tag coupled beads.

The term “end-point analysis” refers to a method where data collection occurs only when a reaction is substantially complete.

The term “real-time analysis” refers to monitoring during PCR. Certain systems such as the ABI 7700 and 7900HT Sequence Detection Systems (Applied Biosystems, Foster City, Calif.) conduct monitoring during each thermal cycle at a predetermined or user-defined stage in each cycle. Real-time analysis of PCR with FRET probes measures fluorescent dye signal changes from cycle-to-cycle, preferably minus any internal control signals.

ID-Tag Probe Complexes

Disclosed are various compositions of ID-tag complexes and methods of using such ID-tag complexes. In order to facilitate the description of such complexes and their use, the description is divided into two parts, “indirect ID-tag complexes” and “direct ID-tag complexes.” However, as will be appreciated by one of skill in the art in light of the present disclosure, there are steps and elements that apply to both.

Indirect ID-Tag Complexes

In one embodiment, the probe complex can detect and indicate the presence of a target sequence even though the target sequence is absent from a final detection system.

One example of such an indirect ID-tag probe complex 101 is shown in FIG. 1A. In this embodiment, the ID-tag complex comprises a probe segment 100 that comprises a probe sequence 110. The probe segment 100 is attached to a first coupling molecule (CM1) 120. This can be at either end of the probe segment; however, the end of the segment that is not hybridized to a probe complement is preferred. The combination of the probe segment 100 and the first coupling molecule 120 is denoted as a probe section 11. Additionally, the indirect ID-tag probe complex 101 further comprises a probe complement section 201 that is hybridized to the probe section 11 via a protruding sequence 111. The probe complement section 201 comprises a probe complement segment 200, which can also be referred to as an ID-tag segment 200. Section 201 also comprises a detectable marker (DM) 130 such as DIG. The probe complement segment 200 comprises a probe complement sequence 111 attached to an ID-tag sequence 115. The probe complement sequence 111 can be shorter than the probe sequence 110 by at least one nucleotide. The probe complement sequence can be shorter by 1-15 or more nucleotides, for example, 1, 2, 3-5, 6-10, 10-15, or more nucleotides, than the probe sequence 110. This, as well as other factors, allows one duplex 50 to be more stable than the other duplex 102.

The ID-tag sequence 115 can be any form of ID-tag sequence as long as its interaction with the probe segment is minimal or does not substantially promote hybridization between the two segments.

In the complex 101, (as shown in FIG. 1A), the probe complement section 201 is hybridized to the probe section 11 via the probe sequence 110 and the probe complement sequence 111. It is through this probe complement sequence 111 that a duplex 102 is formed between part of the probe sequence 110 and the probe complement sequence 111. In one embodiment, the length of the probe complement sequence 111 is shorter than the probe sequence by at least one nucleic acid. While the duplex 102 is stable when there is no target sequence 10 present, the probe sequence comprises a sequence that is complementary to the target sequence as well as the probe complement sequence. In other words, the probe sequence 110 can bind to both the probe complement sequence and the target sequence. Thus, the presence of a target sequence will dissociate the initial duplex 102 and promote the formation of a new duplex 50. This target dependent disruption of the initial duplex 102 is what allows one to later detect the presence or absence of a target sequence.

The interaction between the probe sequence 110 and the probe complement sequence 111 can be weaker than the interaction between the probe sequence 110 and the target sequence 10 in a number of ways, for example, the differences in length of the hybridized duplex formed between the probe complement sequence 111 and probe sequence 110 and the duplex between the probe sequence 110 and target sequence 10. Alternatively, the probe sequence 110 can comprise PNA, the probe complement sequence 111 can comprise DNA, and the target sequence 10 can comprise miRNA. As the interaction between PNA and miRNA is more favored than the interaction between DNA and PNA, this can also result in a target dependent hybridization event that is relatively irreversible.

The addition of the ID-tag probe complex 101 to a sample with a suitable target for the probe sequence 110 results in the binding of the probe sequence 110 to the target sequence 10, as shown in FIG. 1B. Once the probe sequence 110 binds to the target sequence 10, a second coupling molecule (CM2) 121 can be added to the sample. The CM2 121 can bind to the CM1 120 that is attached to the probe segment 100. Following this, the CM2 121 can be removed from the sample, leaving substantially only those segments that lack any CM1 120 or are associated with any CM1 120 (as shown in FIG. 1C). Any device and/or method known in the art to remove the CM2 121 can be used to remove the CM2 and any associated molecules, assuming that the device and/or method does not result in the substantial dissociation of those sequences, segments, and/or sections associated with the CM1 120 and the CM2 121.

Following this, the remaining sample can be added to an array or other detection device 190 that comprises an ID-tag detection sequence 116 that is complementary to the ID-tag target sequence 115 of the probe complex 101. The probe complement section 201 can associate at particular positions on an array, via ID-tag sequence 116 (also known as “detection” ID-tag sequences) that are positioned at various locations 180, on the detection system 190. This paired duplex or hybrid is called a detection complex 151. The ID-tag sequences 116 of the detection system can hybridize to the ID-tag sequence 115 on the probe complement section 201. As the probe complement section 201 comprises a detectable marker 130, one need only look for the presence of the detectable marker 130 at a given array position, e.g., 180, 181, or 182, to determine if there is a ID-tag duplex 114 formed. This can indicate the presence of a target sequence in a sample. Of course, as will be appreciated by one of skill in the art, this application need not be limited to traditional array devices nor need the exact identity of the ID-tag be known while manufacturing the system.

The following section discusses the method of using the above compositions in greater detail.

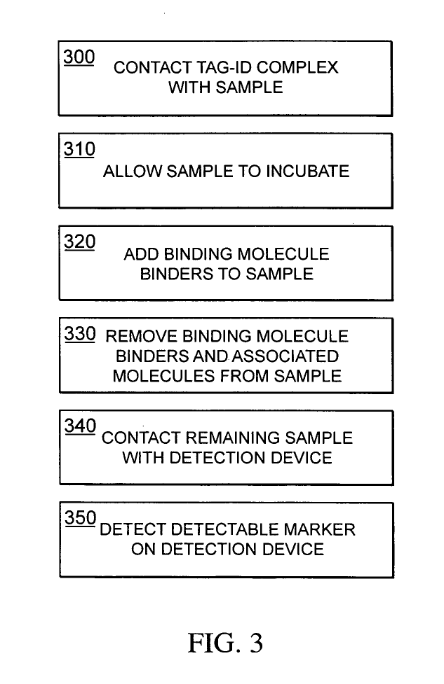

FIG. 3 is a flow chart depicting one embodiment for the method of using the indirect ID-tag complexes described above. In this embodiment, the first step 300 is to add the ID-tag complex 101 to the sample that one wishes to test for the presence of a particular target sequence 10. The particular characteristics of the ID-tag complex can depend, as described herein, upon the characteristics of the target sequence. The amount of the ID-tag complex 101 added can be more than the amount of the target sequence 10 present in the sample. The probe sequence 110 can be comprised of PNA and the probe complement sequence can be comprised of L-DNA, which will provide additional advantages to the probe complex 101 in its detection and hybridization to the target sequence, assuming that the target sequence is not also L-DNA. In some embodiments, it is desirable that the interaction between the probe sequence 110 and the probe complement be such that the only effective way that they will separate is by the presence of the target sequence 10.

The next step 310 involves waiting for a sufficient time, so as to allow the ID-tag complex 101 to dissociate and allow the probe sequence 110 to bind to the target sequence 10 to create a target-probe complex 51 or duplex 50. As this can be the favored duplex, the conditions for such dehybridization and rehybridization can be varied as desired, to achieve speed or reliability accordingly. However, in some embodiments, the conditions are optimized so that dehybridization of the complex 101 is minimized, unless a target sequence is present in the sample. In other words, the conditions can promote the stability of the complex 101 and it is the presence of the target sequence 10 that is responsible for the dissociation of the duplex 102.

In the third step 320, after allowing the formation of the second complex 51, CM2 121 is added to the sample and allowed to bind to the CM1 120. This step can involve the addition of streptavidin beads if, for example, the CM1 120 is Biotin. As will be appreciated by one of skill in the art, the precise identity of the CM1 and CM2 is not crucial, as long as the two form an interaction of sufficient specificity and strength so as to allow the fourth step to be carried out. In one embodiment, the CM1 and CM2 is a second pair of ID-tag sequences.

In the fourth step 330, after the CM2 121 and CM1 120 have bound to one another, one can remove the CM2 121 and anything associated with it from the sample. As shown in FIG. 1B, this can include all of the probe-target complex 51 and the ID-tag complex 101. Thus, after the removal of the CM2 121, all segments and/or sequences directly bound to a CM1 120 and indirectly bound to a CM1 (e.g., through hybridization) will be removed from the sample. This leaves a sample that can contain, as concerns the relevant sequences, primarily, or only, the probe complement section 201. In some embodiments, the process through this point can be kept under conditions such that the separation of the probe complex 101 will not occur or can be minimized unless the separation is initiated by the binding of a target sequence 10 to the probe sequence 110. This initiation can occur at the probe sequence overhang 113, which can provide a location for the target sequence to initially bind, while allowing the probe complex 101 to be maintained unless further hybridization between the probe sequence and the target sequence occurs.

The next step 340 involves taking the remaining sample, as shown in FIG. 1C, and applying it to a detection system, as shown in FIG. 2. This detection system can be one of any number of systems known in the art, as long as it is capable of supporting an ID-tag sequence 116 that is complementary to an ID-tag sequence 115 of the probe complement section 201. In one embodiment, a detection system 190 is used which employs multiple different ID-tag sequences 116, each at a different position, 180, 181, and 182 for example, on an array. As will be appreciated by one of skill in the art, the hybridization conditions can vary based on possible parameters of the sample. For example, where there is little chance that any sequence in the sample can bind to the ID-tag sequence 116, then hybridization conditions can be very favorable. On the other hand, if there may be contaminants that might be able to bind to the ID-tag sequence 116, then the conditions can be modified to reduce the risk of nonspecific binding. Of course, nonspecific binding need not lead to false direct results, as the contaminants can lack any detectable marker that is similar to the section's 201 detectable marker 130.

Referring to FIGS. 2 & 3, one then detects 350 the detectable marker 130. When this is done on an addressed array and with a fluorescent marker as the detectable marker 130, one need only look to the array to see which location, for example 180, 181, 182 is exhibiting fluorescence to determine if the target sequence or sequences were present in the sample. Of course, if more than one different target sequence is present in the sample and more than one probe sequence 110 and ID-tag sequences 115 and 116 used, then multiple positions can be scanned for fluorescence simultaneously in order to detect the presence of the multiple target sequences. The presence of a particular target sequence along with several additional but different target sequences can be examined through the use of different ID-tag sequences being associated with each different probe sequence through the probe complement sequence. Since each probe sequence is associated to a particular ID-tag sequence (through a probe complement sequence) then the presence of a particular target sequence will result in only a particular ID-tag sequence remaining in the sample throughout steps 300-330. Additionally, as each probe complement section 201, 221, and 231 has a particular ID-tag sequence (115, 125, and 135) that can bind significantly to its complement (116, 126, and 136), then the presence or absence of particular target sequence and/or sequences can be examined and resolved simultaneously, even when using a single type of detectable marker, assuming that one has a way to identify which ID-tag sequence 116, 126, or 136 is located at a particular position.

Aspects of the present teachings may be further understood in light of the following examples, which should not be construed as limiting the scope of the present teachings in any way.

### EXAMPLE 1

### Indirect ID-Tag Complexes

This example provides a demonstration of how an indirect ID-tag complex can be used to detect a miRNA sequence in a sample from a patient. One collects a sample from a patient. An indirect ID-tag complex comprises 1) biotin connected to a PNA probe sequence and 2) a L-DNA probe complement sequence, a L-DNA ID-tag target sequence, and DIG (digoxigenin) connected in an appropriate fashion. The complex 1) and 2) is initially hybridized together, as shown in FIG. 1A. This ID-tag complex is contacted with the sample under conditions such that the ID-tag complex can remain hybridized unless a more favorable binding sequence, e.g., the target sequence, is present in the sample. The ID-tag complex is added in excess of the estimated amount of the target sequence. After allowing enough time to pass for a probe-target duplex to form, an excess of streptavidin is added to the sample under conditions that allow for the binding of streptavidin to biotin, but minimize the dissociation of the duplexes formed. Following this, the streptavidin is removed from the sample, removing any molecules associated with it through its binding to biotin. Following this, the sample is applied to an array, the array comprising an ID-tag detection sequence that is complementary to the ID-tag target sequence. The ID-tag detection sequence is located at a first location. After washing the array, one then scans the array for the presence of DIG. In particular, the presence of DIG at the first location indicates the presence of the target sequence in the sample. The amount of DIG at the first location indicates the amount of target sequence present in the sample.

Direct ID-Tag Complexes

In one embodiment, the probe complex can detect and display the presence of a target sequence while optionally retaining the target sequence on the final detection system. One such example is shown in FIGS. 4A through 4C and FIG. 5.

As illustrated in FIGS. 4-5, the ID-tag complex comprises a probe complement section 601 and a probe (ID-tag) section 511. The probe section 511 further comprises a detectable marker 530 and a segment that comprises a probe sequence 510 that can hybridize to a target sequence 10; the probe section 511 further comprising an ID-tag sequence 615 that can hybridize to a complementary ID-tag sequence 116. The two sequences 510 and 615 can be linked by a linker 570. The probe complement section 601 comprises a first coupling molecule (CM1) 520, such as biotin, for example, and a probe complement segment 600. The probe complement segment 600 comprises a probe complement sequence 611.

As will be appreciated by one of skill in the art, the general relationship between the probe sequence/segment and the probe complement sequence/segment as concerns their target sequence dependent hybridization characteristics can be similar between the direct and the indirect ID-tag complexes. For example, the probe complement sequence 611 and the probe sequence 510 are able to hybridize together in a substantially stable manner until the exposed section of the probe section 513 begins to hybridize to the target sequence 10. Upon this binding, the probe complement sequence 611 and the probe sequence 510 unhybridize, break, or dissociate and the probe sequence 510 and target sequence 10 are able to fully hybridize. This results in a target-probe duplex 550, as shown in FIG. 4C.

Once this target-probe duplex 550 has been formed, a second coupling molecule (CM2) 621 can be added to a sample that contains the CM1 520, resulting in the association of the CM2 621 and the CM1 520, as shown in FIG. 4B. This will associate the CM2 621 with all of the probe complement sequence 611 and allow any ID-tag complex 501 that has not dissociated, and any free probe complement sections 601 to be removed from a sample by removing the CM2 621.

The target-probe complex 551 can remain in the sample after any CM2 621 is removed because the target-probe duplex lacks any remaining associated CM1 520. The sample containing the complex 551 can then be added to a detection system 190. This detection system, such as an array, can comprise ID-tag sequences 116 (also known as detection ID-tag sequences) that are complementary to the ID-tag sequence 615 of the ID-tag complex 501. The ID-tag sequence 615 of the target-probe complex 551 that remained in the sample can then bind to the complementary ID-tag sequence 116 in of the detection system 190, which can be located at a particular position 180, for example, on an array. The binding of the probe-target complex to the ID-tag sequence 116 results in the formation of a detection complex 651. In some embodiments, the detection complex 651 contains the target sequence 10.

One can then check the array for the presence of the detectable marker 530. The presence of the detectable marker 530 will indicate that a target sequence 10 and target segment 1 was present in the sample.

The presence of a detectable marker at a particular location, for example 180, instead of another location, for example 181, can indicate the sequence and identity of the particular target sequence that was present in the sample. This can be achieved by indexing particular ID-tag sequences that form particular ID-tag duplexes 114 at particular locations. For example, at one array position 181 there is a particular ID-tag detection sequence 700. This sequence 700 can effectively only bind to a target ID-tag sequence 701, which is associated with a particular probe sequence 710 that will bind to a particular target sequence 711. At a second position 182, there is a different ID-tag detection sequence 702 that can effectively only bind to a target ID-tag sequence 703, which is associated with a particular probe sequence 712 that will bind to a particular target sequence 713. Thus, by examining an array, or other detection system, for the presence of a detectable marker 530 at a particular location, e.g., 181 or 182, one is able to determine if one of or both of the target sequences and segments 711 and 713 are present in the sample by determining if a detection complex 661 or 671 has been formed. As will be appreciated by one of skill in the art, the number of positions and possible target sequences to be examined can be increased to very large numbers. As will be appreciated by one of skill in the art, the above can apply to both direct and indirect complexes.

As will be appreciated by one of skill in the art, the target sequence need not actually be present in the final array or detection system in every embodiment for it to be a direct ID-tag method or complex. In some embodiments, a detection complex 651, 661, or 671 is created and the probe section 511 is later removed. Alternatively, the probe-target complex 551 can be created and later dissociated before it is applied to the detection system. Alternatively, following the removal of effectively all of the ID-tag complex 501 from a sample, any remaining probe sections 511 can be tested on the detection system and the presence of a DM still indicate the presence of a target sequence in the sample. If, for some reason, one needs to distinguish between direct and indirect ID-tag complexes or methods, the distinction does not lie in the final detection complex formed, but rather whether the piece retained was directly bound to the target sequence (direct) or relates to the target sequence through an additional piece (indirect).

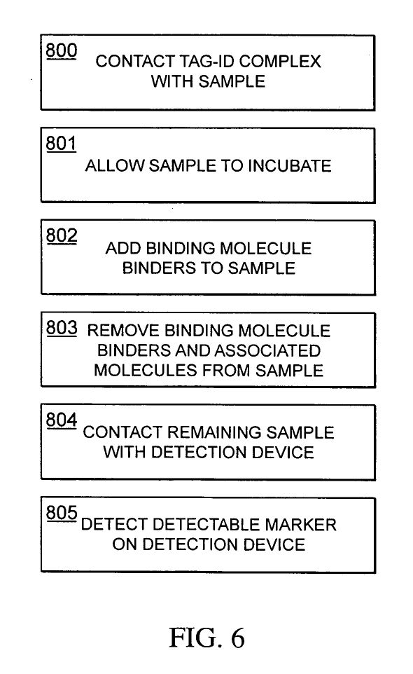

The following section and FIG. 6 describe, in greater detail, the method involved in examining a sample for a particular target sequence using an ID-tag complex 501.

A flow chart for one embodiment of using the direct ID-tag complex 501 is shown in FIG. 6. In this embodiment, the first step 800 involves contacting the ID-tag probe complex 501 with the sample (FIG. 4A). The next step 810 involves waiting a sufficiently long period of time to allow the ID-tag probe complex 501 to dissociate and create a new probe-target 551 complex. As was described above, the extra piece 513 of the probe sequence 510 that is not initially hybridized to the probe complement sequence 611 can initiate the hybridization of the probe sequence 510 to the target sequence 10. When the probe sequence is PNA and the probe complement is L-DNA, an additional advantage will be obtained as the initial hybridization of the sequence 513 will help initiate the disassociation between the probe complement 611 and the probe sequence 510. Additionally, in situations where the probe complement is DNA and the probe sequence 510 is PNA, the target-probe duplex 550 will be even more favored as the PNA-RNA duplex is more stable than the PNA-DNA duplex. Finally, the longer the length of the additional section 513, the more biased the target-probe duplex 550 will be.

The conditions under which this occurs can be varied to fit the particular circumstances and the desired features to be optimized by the search. However, generally, one may select conditions to allow the separation of the ID-tag complex 501, when the target sequence 10 binds to the probe sequence 510. In other words, dissociation of the two sections 601 and 511 is effectively infrequent. As will be appreciated by one of skill in the art, this can depend upon the level of acceptable noise in the assay and level of sensitivity desired.

After waiting a sufficient amount of time to allow the formation of a substantial amount of target-probe duplex 550, the solution can be treated with a second coupling molecule (CM2) 621, such as streptavidin for example, as shown in FIG. 4B and in FIG. 6 step 820. In some embodiments, the amount of CM2 621 added and the conditions under which it is allowed to bind to the CM1 520 are such that substantially all of the CM1 520, and those molecules then associated with the CM1, such as the probe complement segment 600 and the probe segment 500 that are part of an ID-tag complex 501 (i.e., the ID-tag complex 501 that did not dissociate due to the binding of a target sequence 10 to the probe sequence 510) are sufficiently associated with the CM2 621. A sufficient degree of association can be determined by one of skill in the art and will be guided by the desire to remove as much of the ID-tag complex 501, which could result in false results if it remains in the sample and proceeds to the detection system. This process will also allow the association of the probe complement segment 600 with the CM2 621; however, this interaction is usually not as relevant.

A sufficient amount of the CM2 621 should be added to guarantee that effectively all of the ID-tag complex 501 has been associated with a CM2 via the CM1 on the probe complement section 601. The amount can vary based on application, and can be, for example, 50%-100%, 100-200, 200-400, 400-600% or more of the amount of ID-tag complex 501 originally added in the first step.

Following the addition of the CM2 621, the CM2 is then removed from the sample 830. As with the addition of the CM2, the removal of CM2 from the resulting sample can be just as important and the sample can be just as free from remnants of the CM2. The precise method of removal of the CM2 can vary depending upon the nature of the CM2 and CM1, although one of skill in the art will be able to determine the most appropriate method given the present disclosure. This step will result in the removal of any molecule presently associated with a first coupling molecule, be it associated covalently or through a hybridized sequence. This step can result in the removal of a significant amount of the probe section 511 that has not bound to a target sequence 10. What is significant can depend upon the particular application; however, removal of 0-100% can be significant, for example, removal of 0-1, 1-3, 3-10, 10-30, 30-50, 50-70, 70-90, 90-99, 99-100 percent of the ID-tag complex 501 can be significant. This step can also result in the removal of the probe complement. In one embodiment, at the end of this step, all of the probe complement segments 600, and anything hybridized thereto, can be removed from the sample. In another embodiment, the only probe segment remaining in the sample will be that probe segment that is not associated with a first coupling molecule, for example, the probe (ID-tag) segment 500, as shown in FIG. 4C. In another embodiment, only those probe (ID-tag) segments that are also associated with a target sequence 10 will remain in the sample.

Referring to FIGS. 5 and 6, in the next step 840, one adds the remaining sample to the detection system 190, such as an array of ID-tag sequences 116 that are complementary to the ID-tag sequence 615 of the ID-tag complex 501. There can be multiple different complementary ID-tag sequences 615 (also known as ID-tag detection sequences, which is part of the ID-tag detection segment, which is part of the ID-tag detection section) on an array, each sequence 700 and 702 designed to specifically bind to a particular complementary sequence 701 and 703 respectively and each sequence 700 and 702 located at a particular position 181 and 182 on an array. One can then wash away all unbound target-probe complex 551. Alternatively, one may employ a system where binding itself leads to a signal from the detectable marker. This may be during the formation of the target-probe duplex 550, in which case an array may not be necessary. Alternatively, this can be at the formation of the ID-tag target sequence 615-ID-tag detection sequence 116 duplex 114, as shown in FIG. 5. This can be achieved, by detection of the DM that is within a certain distance of the ID-tag sequence 116, e.g., through FRET, or within a certain distance of the position on the array, 180, e.g., through FRET or detection of light from a certain focal layer.

In one embodiment, the next step 850 involves the actual detection and optional quantitation of the detectable marker on the detection system. When each ID-tag sequence 701 and 703 are connected to different probe sequences 710 and 712, one is able to use the system to simultaneously detect different target sequences and/or segments in a sample by looking for the presence or absence of a detectable marker at a particular position, e.g., 181 and 182. As will be appreciated by one of skill in the art, this can depend upon the particular type of detectable marker or markers used, the detection system used, and the particular goals of the particular process, among other factors. In one embodiment, fluorescence levels are examined across the various positions on an array, where each of the positions on an array corresponds to a particular ID-tag detection sequence that can bind to a particular ID-tag target sequence that is covalently attached to a particular probe sequence 510. The amount of signal at each position can be detected and compared to a positive control that involves a saturating amount of an ID-tag complex 501 with a probe sequence 510 that can bind to a known amount of added control target segment 1.

As will be appreciated by one of skill in the art, the process described above can be used to detect the amount of target sequence 10 as each sequence can be bound by a probe section 511 which will have its own detectable marker; thus, the amount of detectable marker can indicate the amount of target sequence present. This is similar for the indirect ID-tag complex 101, as each target sequence can result in an additional detectable marker being added to the detection system. Additionally, the direct ID-tag complex can be used to detect the amount of target segment in the sample as well. For example, while there may be multiple probe sections 500 attached to a single target segment 1, under some situations, only one of the ID-tag target sequences can bind to the array system, for example, when there is only one ID-tag detection sequence 116 available on the array. Using the number of detectable markers present for a single (or nonsaturating number) ID-tag detection sequence 116, one can determine the number of target sequences per ID-tag detection sequence 116. This can then be used to determine how many segments there are from an array system where there are numerous ID-tag detection sequences 116.

In one embodiment, the target is miRNA or other similarly sized or problematic target sequence.

### EXAMPLE 2

### Direct ID-Tag Complexes

This example provides a demonstration of how a direct ID-tag complex can be used to detect a miRNA sequence in a sample from a patient. One collects a sample from a patient. A direct ID-tag complex is administered and comprises 1) DIG connected to a PNA probe sequence (which is complementary to the miRNA target sequence), which is connected to a L-DNA ID-tag target sequence, and 2) a L-DNA probe complement sequence connected to biotin.

The complex is initially hybridized together, as shown in FIG. 4A. This ID-tag complex is contacted with the sample under conditions such that the ID-tag complex can remain hybridized unless a more favorable binding sequence, e.g., the target sequence, is present in the sample. The ID-tag complex is added in excess of the estimated amount of the target sequence. After allowing enough time to pass for a probe-target duplex to form, an excess of streptavidin is added to the sample under conditions that allow for the binding of streptavidin to biotin, but minimize the dissociation of the duplexes formed. Following this, the streptavidin is removed from the sample, removing any molecules associated with it through its binding to biotin. Following this, the sample is applied to an array, the array comprising an ID-tag detection sequence that is complementary to the ID-tag target sequence. The ID-tag detection sequence is located at a first location. After washing the array, one then scans the array for the presence of DIG. In particular, the presence of DIG at the first location indicates the presence of the target sequence in the sample. The amount of DIG at the first location indicates the amount of target sequence present in the sample.

Immobilization of a Sequences, Segments and Sections to a Surface:

One or more sequences, segments, and/or sections can be immobilized to a surface for the purpose of creating various arrays. They can be immobilized to the surface using the well known process of UV-crosslinking, for example. Alternatively, the sequence can be synthesized on the surface in a manner suitable for deprotection but not cleavage from the synthesis support. In one embodiment, the ID-tag sequence 116 is immobilized to a surface (FIG. 2 and FIG. 5). In these embodiments, the ID-tag sequence 116 and ID-tag segment can comprise L-DNA. Thus, methods for attaching DNA and L-DNA in particular can be employed. Alternatively, the ID-tag segment can comprise more than just an ID sequence 116, and can comprise additional sequences. In some embodiments, these additional sequences are configured for ease of attachment between the ID-tag segment and the surface. In some embodiments, this can mean an entire sequence is added to the ID-tag segment. In other embodiments, only a single nucleic acid, or a particular functional group, is added to allow the ID-tag segment surface interaction to form.

The sequences, segments, and/or sections can be covalently linked to a surface by the reaction of a suitable functional groups on the probe and support. Functional groups such as amino groups, carboxylic acids and thiols can be incorporated in a sequence, segment, section, and/or complex thereof by extension of one of the termini with suitable protected moieties (e.g. lysine, glutamic acid and cystine). In some embodiments, when extending the terminus, one functional group of an amino acid such as lysine can be used to incorporate the donor or acceptor label at the appropriate position in the polymer (See: PNA Labeling) while the other functional group of the branch is used to optionally further extend the polymer and immobilize it to a surface.

Methods for the attachment of sequences, segments, sections, and/or complexes thereof to surfaces can generally involve the reaction of a nucleophilic group, (e.g. an amine or thiol) of the probe to be immobilized, with an electrophilic group on the support to be modified. Alternatively, the nucleophile can be present on the support and the electrophile (e.g. activated carboxylic acid) present on the analog probe complex. When the item to be attached contains PNA, because native PNA comprises an amino terminus, a PNA segment will not necessarily require modification to thereby immobilize it to a surface (See: Lester et al., Poster entitled “PNA Array Technology”).

Conditions suitable for the immobilization of a PNA sequence to a surface will generally be similar to those conditions suitable for the labeling of a PNA (See discussion of PNA Labeling below). The immobilization reaction is the equivalent of labeling the PNA whereby the label is substituted with the surface to which the PNA probe is to be covalently immobilized.

Numerous types of surfaces derivatized with amino groups, carboxylic acid groups, isocyanates, isothiocyanates, and maleimide groups are commercially available. Non-limiting examples of suitable surfaces include membranes, glass, controlled pore glass, polystyrene particles (beads), silica, and gold nanoparticles.

As will be appreciated by one of skill in the art, and as discussed above, an example of a PNA segment 20 was particularly emphasized above. However, this is by way of convenience, as other nucleotides and analog nucleotides, particularly with similar characteristics can be used. In one embodiment, the ID-tag sequence is attached to a surface via an amino linker, for example by using a 3′-amino-modifier C6 CPG (Glenresearch).

Arrays of ID-Tags:

Arrays are surfaces to which two or more probes of interest have been immobilized. In some embodiments, said immobilization occurs at predetermined locations. Arrays comprising both nucleic acid stereoisomer analog nucleic acids (such as L-DNA) and PNA probes have been described in the literature. The probe sequences immobilized to the array are chosen to interrogate a sample that can contain one or more target sequences of interest. Because the location and sequence of each probe is known, arrays are generally used to simultaneously detect, identify or quantitate the presence and/or amount of one or more target sequences in the sample. Actual detection can be done through any number of devices; for example, a chemiluminescence analyzer can be used (such as the 1700 Chemiluminescent Microarray Analyzer from Applied Biosystems).

Since the composition of the probe and probe complement sections of the detection complex are or can be known because of its location on the surface of the array (e.g., because an ID-tag complex was synthesized or attached to this position in the array), the composition of target sequence(s) can be directly detected, identified and/or quantitated by determining the location of detectable signal generated in the array.

In some embodiments, the arrays can be useful for diagnostic applications and for use in diagnostic devices. The arrays can be used to establish a correlation between the amount of a particular nucleotide sequence (e.g., siRNA, miRNA, etc.) and a disease, including a particular stage of a disease. In a further embodiment, once such a correlation between the amount of a nucleotide sequence and a particular disease, including a particular stage of a disease has been made, or is known, the arrays can be used to diagnose a particular disease, including a stage of a disease in a tissue of an organism. Accordingly, a method of diagnosing a disorder, e.g., disease, in a patient is also contemplated. One or more target sequences that are known to be associated with the disorder from which a patient is believed to be suffering are selected. For example, if a patient is suspected of suffering from a tumor, the methods described herein can be used to identify the presence of one or more RNA or DNA sequences that are known to be expressed in tumor cells, but not in normal cells. Similarly, if a patient is suspected of having been exposed to an infectious agent, nucleotide sequences known to be associated with the infectious agent are selected for identification. For example, a sample from a patient suspected of being infected with HIV may be analyzed for the presence of nucleotide sequences known to be associated with HIV, such as GP120MN.

As will be appreciated by one of skill in the art, while the ID-tag detection sequence is attached to a single position, the position itself may be relocated. This can be done, for example, if the position is on a bead or other particulate material that can be relocated. As will be appreciated by one of skill in the art, the identity of the ID-tag detection sequence can be determined and correlated by particular characteristics and properties of the particles, e.g., beads, themselves.

Detectable and Independently Detectable Moieties/Multiplex Analysis:

In some embodiments, a multiplex hybridization assay is performed. In a multiplex assay, numerous conditions of interest are simultaneously examined. Multiplex analysis relies on the ability to sort sample components, including the data associated therewith, during or after the assay is completed. In one embodiment, different detectable markers are used for ID-tag complexes with different probe sequences. However, as will be appreciated by one of skill in the art, as each different probe sequence can be correlated to a different ID-tag sequence, and each ID-tag sequence can be correlated to a particular position on a detection system, e.g., array, multiple detectable markers are not required.

The ability to differentiate between and/or quantitate the presence or absence of a detectable marker at each of several positions on the detection system provides a means to multiplex a hybridization assay. The hybridization of each distinct set of ID-tag sequences, can be correlated with a distinct probe sequence, which can be correlated with a distinct target sequence or sequences sought to be detected in a sample.

Consequently, these multiplex assays can be used to simultaneously detect the presence, absence, or amount of one or more target sequences, which can be present in the same sample in the same assay. Independently detectable fluorophores can be used as the detectable markers of a multiplex assay using ID tag-complexes if desired to add an additional dimension to the variability.

An example of a possible multiplex analysis follows for an indirect ID-tag system. Two different ID-tag complexes can be used to detect each of two different target sequences. Each ID-tag complex can have a different probe sequence and a different probe complement sequence. Additionally, the ID-tag sequence associated with each probe complement sequence can also be sufficiently different so they do not allow nonspecific ID-tag duplex 114 formation. Finally, at a first position 180 on an array, a first ID-tag detection sequence can be attached and at a second position 181, a second ID-tag sequence can be attached. Consequently, one then monitors the level of the detectable marker, e.g., brightness, at each position on the array. The presence of the detectable marker at any particular location, and relative amount thereof, can indicate the presence of a target sequence in the sample and the relative amount of the target sequence as well. As will be appreciated by one of skill in the art, one may look at multiple positions on the array consecutively, randomly, or simultaneously to determine if more than one target sequence is present in a sample and their relative amounts. As will be appreciated by one of skill in the art, the detection of different sequences can have additional advantages over multiple separate sample examinations, as it can provide for an internal control, e.g, zeroing of the relative amounts of each target sequence, and it can provide for a rapid production of data for many target sequences.

As will be appreciated by one of skill in the art, the arrays, detection systems and multiplexing approaches described above can also easily be used for the direct ID-tag complexes as well.

In one embodiment, the ID-tag complexes are administered in vivo. Following this, a sample is extracted, CM2 added to the sample and then removed, and then the remaining sample applied to an array with ID-tag detection sequences.

### EXAMPLE 3

### Detection of Multiple Target Sequences Simultaneously

This example demonstrates how one can use several ID-tag complexes on a single sample to determine the presence or absence of multiple target sequence. One first creates a detection complex for each target sequence to be detected in a sample. Each complex has an effectively unique probe sequence, probe complement sequence, and ID-tag sequence so that there is little non-specific binding. Given three target sequences, A, B, and C, one creates three ID-tag complexes, A′, B′, and C′ that comprise an “A” probe sequence, an “A” probe complement sequence, and an “A” ID-tag sequence. The B′ and C′ ID-tag complexes are similarly comprised of B and C sequences.

The ID-tag complexes are contacted with the sample under conditions such that the ID-tag complexes can remain hybridized unless a more favorable binding sequence, e.g., one of the target sequence, is present in the sample. The ID-tag complexes are added in excess of the estimated amount of their respective target sequences. After allowing enough time to pass for probe-target duplexes to form, an excess of streptavidin, greater than the sum of all biotin in the sample, is added to the sample under conditions that allow for the binding of streptavidin to biotin, but minimize the dissociation of the duplexes formed. Following this, the streptavidin is removed from the sample, removing any molecules associated with it through its binding to biotin. Following this, the sample is applied onto an array comprising various different ID-tag detection sequences that are complementary to the particular ID-tag target sequences of the complexes. There is an “A” ID-tag detection sequence, a “B” ID-tag detection sequence, and a “C” ID-tag detection sequence. Each type of ID-tag detection sequence is located at a different location; for example, ID-tag detection sequence A is at a first, ID-tag detection sequence B is at a second and ID-tag detection sequence C is at a third location on the array. After washing the array, one then scans the array for the presence of the detectable markers. One looks for the presence of the detectable marker at a particular location. The presence of the detectable marker at the first and third location, in a 2:1 ratio indicates that the sample contains sequence A and sequence C and that there is twice as much sequence A as there is sequence C.

Kits

In some embodiments, such as a kit, the elements are provided but not yet connected. Thus, for example, a kit can comprise one or more first vials of an ID-tag complex 501, 101, a vial with second coupling molecules 621, 121, and one or more vials of a corresponding ID-tag detection sequence or sequences 116, 116′. Alternatively, the kit can comprise a vial comprising a probe segment, a vial comprising a probe complement segment, an array system, such as a bead based system with ID-tag sequences on the beads, and a vial of second coupling molecules. Instructions for performing detection methods can be included in the kits. The kit can comprise a vial which contains DM 530, 130 that is already associated with a segment 500, 200. The kit can contain multiple vials, each with a different DM or DMs associated with a different segment 200 or 500. In one embodiment, the sequences 615 or 115 are already combined into their larger sections 511 or 201.

In one embodiment, the kit comprises two vials, each with half of a set of an ID-tag sequence that together can form a duplex 114. At the end of one of the sequences 615 of each set of sequences, there is a linker 570. The linker can be used to attach various alternative sequences to the ID-tag sequence. For example, it can be used for attachment to a probe sequence 510 or a probe complement sequence 111. The opposite end of the complementary ID-tag sequence 116 can be configured to be attached to a surface (for example through an extra length of sequence). The kit can further comprise a vial of a CM2 and a vial of a CM1. The kit can further comprise a surface upon which the ID-tag sequences can be attached. Alternatively, one of the complements of the ID-tag sequences can already be attached to surface. Of course this can be adjusted appropriately for the direct or indirect ID tag complex and method of use as described herein.

The kit can further comprise various salts or other stringency agents that can be used to alter the ability of the complexes to remain as complexes or form new complexes. The kit can also comprise additional reagents that allow FRET to occur between the detectable marker and the 1) ID-tag detection sequence, 2) the array platform, and/or 3) base to which the ID-tag detection sequence is attached. The kit can comprise various vials or containers for the storage, measurement, mixing, etc., of the various components.

Components:

The sequences involved with the ID-tag-coded complexes, e.g., 10, 110, 111, 115, 116, 510, 615, 611, 181, and 182, in FIGS. 1A-1C, 2, 4A-4C, and 5, can be made from PNA, D-DNA, L-DNA, L-RNA, D-RNA, O-methyl RNA (e.g., 2′ O-methyl), or other types of nucleic acids described above, as long as they are configured so that they perform their described function for a particular embodiment. Thus, the ID-tag-coded complex need not include L-DNA or PNA and need not be chimeric. As will be appreciated by one of skill in the art, in some embodiments, the sequences are placed immediately adjacent to one another to create the segments and sections, thus making them contiguous with each other. In another embodiment, additional spacers are added between each of the sequences and/or segments described herein, resulting in sequences that are not contiguous with one another.

ID-Tag Sequences

By “ID-tag sequence” it is meant that the particular set of sequences creates a sufficiently stable duplex 114, thus, effectively connecting one sequence 115 or 615 to the second sequence 116. This can be done in a variety of ways. For example, selecting the nucleotides to optimize the melting temperature of the duplex 114. A sequence can be an ID-tag sequence if it will not substantially melt from its ID-tag coded complement at degrees Celsius ranges from below 50° to more than 120°, for example, at less than 50, 50-60, 60-70, 70-80, 80-90, 90-100, 100-120 degrees C. or more. A sequence can be an ID-tag when the particular sequence selected will not substantially hybridize to a sequence that the ID-tag sequence will be exposed to. Thus, by knowing the possible sequences in a reaction mixture, for example in an organism, one is able to design a probe that will not hybridize to sequences in the reaction mixture(. The ID-tag sequences 116, 615 and 115 can be made of L-DNA, to create probes that are resistant to cellular degradation through the action of cellular nucleases.

In one embodiment, the “ID-tag sequence” also allows the ready identification of a particular sequence to a particular detectable component. In some of the present embodiments, the ID-tag sequence allows one to index a particular probe sequence with a particular DM. In one embodiment, the ID-tag detection sequence can serve as a marker for identifying itself. For instance, in a randomly assembled array device where the precise location of the ID-tag detection sequences are not initially known, one may add a substance that will bind to the various ID-tag detection sequences in order to determine its identity and what probe or probe complement segment will bind to it.

An ID-tag sequence can be one that, when hybridized to its complement, will not substantially separate during use. For example, some analog sequences, such as L-DNA sequences, will only bind to other L-DNA sequences. Thus, any pair of sequences that are L-DNA sequences can serve as an ID-tag duplex, as long as substantially complementary L-DNA is not in the sample. In situations where sequences exist that can bind to L-DNA, then the above discussed aspects can be included to make the duplex an ID-tag duplex.

The length of the ID-tag sequences 115 and 615, and the sequences 116 that forms the duplex 114, can vary depending upon the use. The ID-tag sequences can be from 1-30 nucleotides, including 4-15, 6-12, or 7 nucleotides long, for example. In one embodiment, the sequences comprise contiguous nucleotides. In another embodiment, variants of the sequences can also be used.

The sequence 510 or 111 and the sequence 615 or 115 can be connected via a chemical moiety that allows flexibility such as a linker 570. The two sequences can be connected by any means which will allow the probes to function. The sequence 510 or 111 and the sequence 615 or 115 can be connected via a PEO/PEO connection, for example. Each of the sequences and/or segments can comprise additional natural or analog nucleotides; thus, for example, not all of the nucleotides between sequences 510 and 600, or between sequences 110 and 111 need to hybridize together. The only requirement is that the relevant duplexes are sturdy enough for the probe to function for its intended purpose.

In one embodiment, the segments 500 and 100 only contain PNA sequences. As will be appreciated by one of skill in the art from the present disclosure, the segment 600 or 200 can comprise a complementary PNA sequence, a complementary L-DNA, L-RNA sequence, as well as other types of natural and analog nucleotides.

As will be appreciated by one of skill in the art, the ID-tag detection sequence can be part of a segment and a section accordingly. The ID-tag detection sequence can be all of or just part of the segment or section. The relationship between sequence, segment and section is similar to that for the other sequences. For example, additional linkers can be added to the ID-tag detection sequence to connect the ID-tag detection sequence to an array. Alternatively, additional sequences, detectable marker, or marker modifiers can also be added to the ID-tag detection sequence. Thus, a detection segment or ID-tag detection segment is a segment comprising an ID-tag sequence.

Detectable Markers

As discussed above, the detectable marker, can be any component which is observable, either directly, for example through fluorescence or MRI, or indirectly, for example through antibody binding and subsequent detection of the bound antibody.

The DM 130 can be positioned at the end of the sequence 111 away from the first coupling molecule 120. Alternatively, the DM 130 can be attached to the sequence 111 at the end closest to the first coupling molecule 120.

The DM 530 can be positioned at the end of the sequence 510 next to the first coupling molecule 520 or at the end near the linker 570. Alternatively, the DM 530 can be attached to the sequence 615 at either end, although synthesis of the section 511 would more challenging.

The sequence 510, 615, 111, and/or 115 can comprise nucleotides that fluoresce, such as analog nucleotides. This can remove or reduce the need for an independent DM. The DM can be inherent in the sequence 510, 615, 111, and/or 115.

As discussed herein, the DM can comprise any detectable molecule. Examples of DMs include quantum dots (Q-dots) or any fluorophore in general. There can be situations involving multiple DMs in a single solution, in such cases, FRET can be employed, assuming the distances between the detectable markers is appropriate for revealing the desired information. Thus, FRET appropriate DMs can be employed.

Probe Sequences

The probe sequence is the sequence recognition portion of the construct. The probe sequence can be designed to hybridize to at least a portion of the target sequence. The majority of the probe sequence can hybridize to the target sequence, or the entire length of the probe sequence can hybridize to the target sequence. In other embodiments, the probe sequence hybridizes to more of the target sequence than the probe complement sequence.

The probe sequences 510 and 110 can be made of RNA, PNA, or any substance that binds to the target sequence more strongly than the probe sequence binds to the probe complement sequence. This need not be on a base by base comparison, and can be based on a length of sequences. Alternatively, the probe sequences 510 and 110 can be made of other analog nucleotides, for example, 2′ O-methyl RNA, 2′ O-ethyl RNA, and 2′ O-ethyl RNA. The probe sequence can be a non-polynucleotide. In one embodiment, the segment 200 or 500 is made of a uniform type of nucleotide or analog thereof. Each sequence of each segment can be a different type of nucleotide or analog thereof. Each sequence can comprise different types of nucleotides or analogs thereof.

The length of the probe sequence (and therefore minimum length of the segment) can be chosen such that a stable complex is formed between the analog probe complex and the target sequence sought to be detected, under suitable hybridization conditions. The probe sequence can be any length, and can depend upon the particular application, as will be appreciated by one of skill in the art. The probe sequence of a PNA oligomer can have a length of between 1 and 40 PNA subunits, including those described above and including 8 to 18, or 8-12 subunits in length. The length of the entire probe segment can also be exactly the same as the length of the target sequence. The length of the probe segment can also be longer than the length of the probe complement sequence by any amount. For example, the PNA segment, and probe segment can be 1, 2, 3, 4, 5, 6, 7, 8, 9, 10, 11-20, 20-30 or greater units longer than the number of nucleic acids present in the probe complement sequence.

The probe sequence can generally have a nucleobase sequence that is complementary to the target sequence. Alternatively, a substantially complementary probing sequence might be used since it has been demonstrated that greater sequence discrimination can be obtained when utilizing probes wherein there exists a single point mutation (base mismatch) between the probing nucleobase sequence and the target sequence (See: Guo et al., Nature Biotechnology 15: 331-335 (1997), Guo et al., WO97/46711; and Guo et al., U.S. Pat. No. 5,780,233, hereby incorporated in their entireties by reference).

The length of the overhang 513, 113 can also vary depending upon the particular embodiment. For example, the overhang can be from 5-10, 3-6, 6, or 7 bases in length. The overhang can also be longer as well.

Probe Complement Sequence

As will be appreciated by one of skill in the art, the probe complement sequence can involve the same variables as the probe sequence. Thus, the discussion concerning aspects of the probe sequence is applicable here as well, with the exceptions that are unique to the probe complement sequence.

In particular, the probe complement sequence can bind to the probe sequence, and vice versa. However, the probe complement sequence can bind to the probe sequence more weakly than the probe sequence can bind to the target sequence. This can be achieved in various ways, e.g., sequence selection, the types of nucleic acids or analogs thereof selected, lengths of the various binding sequences, etc.

Additionally, while the probe-target duplex is relatively stable, the probe complement-probe duplex can be less stable, as it can be broken in order for the probe-target duplex to be formed.

Additionally, while the probe sequence can bind to the target sequence, in one embodiment, the probe complement sequence does not substantially bind to the target sequence or any non-probe sequence in the sample. As will be appreciated by one of skill in the art, this can be achieved in a variety of ways. The probe complement sequence can comprise L-DNA, and the sample can lack any substantial amount of complementary L-DNA. In such an embodiment, the probe sequence can comprise PNA or any nucleotide analog that can bind to both the target sequence and L-DNA. In another embodiment, the probe complement is a D-DNA sequence. The target sequence can be a RNA sequence and the probe sequence can be a RNA sequence or some analog thereof so that the probe-target duplex will be a RNA-RNA or analog thereof duplex. Of course, given the present disclosure, one of skill in the art could select alternative arrangements that can also perform as desired.

The targeted sequence can be any nucleotide, including a set of nucleic acids. For example, DNA, rRNA, or mRNA can be targeted. The target sequence can be from any source, e.g., genomic or cDNA, and can include analog or artificial nucleic acids as well. The ID-tag complexes (IDTCs) can be used to determine the amount of target sequence present in a sample.

Typically, cellular uptake of PNA is inefficient because the back bone has a neutral charge. A chimeric PNA and L-DNA ID-tag-coded complex should increase cellular update since L-DNA has indirect charges, as does normal D-DNA. In addition, the stereochemistry of L-DNA ensures that it will not hybridize to mRNA and gDNA in cells; such a reaction would interfere with the detection procedure.

Some features of some of the embodiments described above can include (1) the cellular uptake efficiency will increase for chimeric PNA/L-DNA probes, (2) L-DNA sequences will not interact with mRNA and gDNA in cells, and (3) the ability to detect very short sequences and/or short segments in situations where traditional approaches may not be successful because of the difficult. However, as will be appreciated by one of skill in the art, not every embodiment will have all or any of these features, and they can have other features as well.

As will be understood by one of skill in the art, the number of ID-tag coded complexes is not limited to 2 at a time. For example, in some embodiments, there can be 3, 4, 5-10, 10-20, 20-40, 40-100, thousands or more of the ID-tag coded complexes. Likewise, as will be appreciated by one of skill in the art, the number of DMs on a single ID-tag coded complex is not limited to only one. For example, there may be 2, 3, 4, 5, 6-10, or any number of DMs, as long as the segments and sequences can function as described. One possible reason to have multiple DMs is that it will allow a greater degree of customization of the signature of the DM. In one embodiment, additional DM and/or Marker Modifiers (MM, such as in FRET based systems) are included on various sequences or segments in addition to that described above to allow for additional distinctions between segments to be made. For example, a DM 130 could be added to probe segment 110 so that the two DMs are next to each other and can undergo a FRET interaction. Alternatively, DMs may be added to the complementary and/or detection ID-tag sequence, to allow a FRET interaction to be the interaction observed as an indicator of the presence of a target sequence. In one embodiment, the MM is also the CM1. Thus, a change in FRET can occur as the probe and probe complement separate from one another upon the binding of the probe to the target. Thus, the complex can be self-indicating for the presence of the target. As will be appreciated by one of skill in the art, the additional DM may be freely moved throughout various positions of the sequences and segments according to the knowledge of one of skill in the art.

PNA Synthesis:

Methods for the chemical assembly of PNAs are well known (See: U.S. Pat. Nos. 5,539,082, 5,527,675, 5,623,049, 5,714,331, 5,736,336, 5,773,571 or 5,786,571 (all of which are hereby incorporated, in their entireties, by reference). Chemicals and instrumentation for the support bound automated chemical assembly of Peptide Nucleic Acids are now commercially available. Chemical assembly of a PNA is analogous to solid phase peptide synthesis, wherein at each cycle of assembly the oligomer possesses a reactive alkyl amino terminus which is condensed with the next synthon to be added to the growing polymer. Because standard peptide chemistry is utilized, natural and non-natural amino acids are routinely incorporated into a PNA oligomer. Because a PNA is a polyamide, it has a C-terminus (carboxyl terminus) and an N-terminus (amino terminus). For the purposes of the design of a hybridization probe suitable for antiparallel binding to the target sequence (the preferred orientation), the N-terminus of the probing nucleobase sequence of the PNA probe is the equivalent of the 5′-hydroxyl terminus of an equivalent DNA or RNA oligonucleotide. An example of PNA is shown in FIG. 7.

L-DNA Synthesis

L-form and D-form phosphoramidite nucleosides can be prepared and used in oligonucleotide synthesis according to known procedures and methods of sugar and nucleobase protection and phosphitylation of the respective nucleosides. D-form nucleosides are derived from naturally occurring D-DNA sources. L-form phosphoramidite nucleosides can be prepared by any suitable synthetic method. For example, L-form phosphoramidite nucleosides can be prepared from L-ribose, which can be derived from L-xylose in a series of steps (Chu, U.S. Pat. No. 5,753,789; Fujimori Nucleosides & Nucleotides 11:341-49 (1992); Beigelman, U.S. Pat. No. 6,251,666; Furste, WO 98/08856).

L-DNA and PNA can be covalently connected in any number of ways, including, for example, via a polymer linker, such as PEO to PEO. In another embodiment, the L-DNA and PNA linked molecule can be produced directly on a synthesizer. A comparison of L-DNA and D-DNA is shown in FIG. 8.

Labels—Detectable Markers and Methods of Attachment

General labeling can be accomplished using any one of a large number of known techniques employing known labels, linkages, linking groups, reagents, reaction conditions, and analysis and purification methods. Detectable Markers (e.g., labels) include light-emitting or light-absorbing compounds which generate or quench a detectable fluorescent, chemiluminescent, or bioluminescent signal (Kricka, L. in Nonisotopic DNA Probe Techniques, Academic Press, San Diego, pp. 3-28 (1992)). Fluorescent reporter dyes useful for labeling biomolecules include fluoresceins (for example, U.S. Pat. Nos. 5,188,934; 5,654,442; 6,008,379; 6,020,481), rhodamines (for example, U.S. Pat. Nos. 5,366,860; 5,847,162; 5,936,087; 6,051,719; 6,191,278), benzophenoxazines (for example, U.S. Pat. No. 6,140,500), energy-transfer dye pairs of donors and acceptors (for example, U.S. Pat. Nos. 5,863,727; 5,800,996; 5,945,526), and cyanines (for example, Kubista, WO 97/45539), as well as any other fluorescent label capable of generating a detectable signal. Specific examples of fluorescein dyes include 6-carboxyfluorescein; 2′,4′,1,4,-tetrachlorofluorescein; and 2′,4′,5′,7′,1,4-hexachlorofluorescein (e.g., U.S. Pat. No. 5,654,442). Another class of labels is hybridization-stabilizing moieties which serve to enhance, stabilize, and/or influence hybridization of duplexes, e.g. intercalators, minor-groove binders, and cross-linking functional groups (Blackburn, G. and Gait, M. Eds. “DNA and RNA structure” in Nucleic Acids in Chemistry and Biology, 2^{nd} Edition, (1996) Oxford University Press, pp. 15-81). Yet another class of labels effects the separation and/or immobilization of a molecule by specific or non-specific capture, for example biotin, digoxigenin, and other haptens (Andrus, “Chemical methods for 5′ non-isotopic labelling of PCR probes and primers” (1995) in PCR 2: A Practical Approach, Oxford University Press, Oxford, pp. 39-54). Non-radioactive labelling methods, techniques, and reagents are reviewed in: Non-Radioactive Labelling, A Practical Introduction, Garman, A. J. (1997) Academic Press, San Diego.

Examples of fluorophores are derivatives of fluorescein, derivatives of bodipy, 5-(2′-aminoethyl)-aminonaphthalene-1-sulfonic acid (EDANS), derivatives of rhodamine, Cy2, Cy3, Cy 3.5, Cy5, Cy5.5, texas red and its derivatives. In principle, any fluorophore can be used. Any fluorophore described in the Ninth Edition of the Handbook of Fluorescent Probes and Research Products, (Edited by Richard P. Haugland, (2002) hereby incorporated in its entirety by reference) can be used, with particular emphasis on the fluorescent molecules in chapter 1. Though the previously listed fluorophores might also operate as acceptors, the acceptor moiety can be a quencher moiety. The quencher moiety can be a non-fluorescent aromatic or heteroaromatic moiety. For example, the quencher moiety can be 4-((-4-(dimethylamino)phenyl)azo)benzoic acid (dabcyl).

Examples of possible methods for attaching a fluorescent probe to a nucleic acid are also provided in the Handbook of Fluorescent Probes and Research Products, Ninth Edition, with special emphasis given to chapter 8, sections 8.1, “nucleic acid stains,” and section 8.2, “labeling oligonucleotides and nucleic acids.” Additionally, labels can be attached though sulfur groups to maleimide groups. Alternatively, labels are attached through additional linkers, such as streptavidin to biotin, which is connected to the nucleic acid sequence, or via a dye-labelled antibody. Thus, the attachment of the label to the segment can be either covalent or noncovalent.

Chemical labeling of a PNA segment and/or sequence can be analogous to peptide labeling. Because the synthetic chemistry of assembly is essentially the same, any method commonly used to label a peptide can be used to label a PNA segment and/or sequence. For example, the N-terminus of the polymer is labelled by reaction with a moiety having a carboxylic acid group or activated carboxylic acid group. One or more spacer moieties can optionally be introduced between the labeling moiety and the probing nucleobase sequence of the oligomer. Generally, the spacer moiety is incorporated prior to performing the labeling reaction. However, the spacer can be embedded within the label and thereby be incorporated during the labeling reaction.

The C-terminal end of the probing sequence can be labelled by first condensing a labelled moiety with the support upon which the PNA is to be assembled. Next, the first synthon of the probing nucleobase sequence can be condensed with the labelled moiety. Alternatively, one or more spacer moieties can be introduced between the labelled moiety and the oligomer (e.g. 8-amino-3,6-dioxaoctanoic acid). Once the segment is completely assembled and labelled, it is cleaved from the support deprotected and purified using standard methodologies.

The labelled moiety can be a lysine derivative wherein the epsilon-amino group is modified with a donor or acceptor moiety. For example, the label could be a fluorophore such as 5(6)-carboxyfluorescein or a quencher moiety such as 4-((4-(dimethylamino)phenyl)azo)benzoic acid (dabcyl). Condensation of the lysine derivative with the synthesis support would be accomplished using standard condensation (peptide) chemistry. The alpha-amino group of the lysine derivative would then be deprotected and the probing nucleobase sequence assembly initiated by condensation of the first PNA synthon with the alpha-amino group of the lysine amino acid. As discussed above, a spacer moiety could optionally be inserted between the lysine amino acid and the first PNA synthon by condensing a suitable spacer (e.g., Fmoc-8-amino-3,6-dioxaoctanoic acid) with the lysine amino acid prior to condensation of the first PNA synthon of the probing nucleobase sequence.

Alternatively, a functional group on the assembled, or partially assembled, polymer can be labelled with a donor or acceptor moiety while it is support bound. This method requires that an appropriate protecting group be incorporated into the oligomer to thereby yield a reactive functional to which the donor or acceptor moiety is linked, but has the advantage that the label (e.g., dabcyl or a fluorophore) can be attached to any position within the polymer including within the probing nucleobase sequence. For example, the epsilon-amino group of a lysine could be protected with a 4-methyl-triphenylmethyl (Mtt), a 4-methoxy-triphenylmethyl (MMT) or a 4,4′-dimethoxytriphenylmethyl (DMT) protecting group. The Mtt, MMT, or DMT groups can be removed from PNA (assembled using commercially available Fmoc PNA monomers and polystyrene support having a PAL linker; PerSeptive Biosystems, Inc., Framingham, Mass.) by treatment of the resin under mildly acidic conditions. Consequently, the donor or acceptor moiety can then be condensed with the epsilon-amino group of the lysine amino acid. After complete assembly and labeling, the polymer is then cleaved from the support, deprotected and purified using well known methodologies.

The DM can be attached to the polymer after it is fully assembled and cleaved from a support. This method is useful where the label is incompatible with the cleavage, deprotection or purification regimes commonly used to manufacture the oligomer. The PNA can generally be labelled in solution by the reaction of a functional group on the polymer and a functional group on the label. Those of ordinary skill in the art will recognize that the composition of the coupling solution will depend on the nature of oligomer and the donor or acceptor moiety. The solution can comprise organic solvent, water or any combination thereof. Generally, the organic solvent will be a polar non-nucleophilic solvent. Non-limiting examples of suitable organic solvents include acetonitrile, tetrahydrofuran, dioxane, methyl sulfoxide and N,N′-dimethylformamide.

Generally the functional group on the polymer to be labelled can be an amine and the functional group on the label can be a carboxylic acid or activated carboxylic acid. Non-limiting examples of activated carboxylic acid functional groups include N-hydroxysuccinimidyl esters. In aqueous solutions, the carboxylic acid group of either of the PNA or label (depending on the nature of the components chosen) can be activated with a water soluble carbodiimide. The reagent, 1-(3-dimethylaminopropyl)-3-ethylcarbodiimide hydrochloride (EDC), is a commercially available reagent sold specifically for aqueous amide forming condensation reactions.

Labelled chimeric configurational oligonucleotides can be formed by coupling a reactive linking group on a label, e.g., a quencher moiety, with the chimeric configurational oligonucleotide in a suitable solvent in which both are soluble or appreciably soluble, using methods well-known in the art. For labelling methodology, see Hermanson, Bioconjugate Techniques, ((1996) Academic Press, San Diego, Calif. pp. 40-55, 643-71; Garman, 1997, Non-Radioactive Labelling: A Practical Approach, Academic Press, London. Crude), labelled chimeric configurational oligonucleotides can be purified away from any starting materials and/or unwanted by-products, and stored dry or in solution for later use, preferably at low temperature.

The label can bear a reactive linking group at one of the substituent positions, e.g., an aryl-carboxyl group of a quencher, or the 5- or 6-carboxyl of fluorescein or rhodamine, for covalent attachment through a linkage. The linkage that links a label to a chimeric configurational oligonucleotide preferably should not (i) interfere with hybridization affinity and/or specificity, (ii) diminish quenching, (iii) interfere with primer extension, (iv) inhibit polymerase activity, (v) adversely affect the fluorescence, quenching, capture, or hybridization-stabilizing properties of the label, (vi) and/or any combination of the foregoing. Electrophilic reactive linking groups form a covalent bond with nucleophilic groups such as amines and thiols on a polynucleotide. Examples of electrophilic reactive linking groups include active esters, isothiocyanate, sulfonyl chloride, sulfonate ester, silyl halide, 2,6-dichlorotriazinyl, phosphoramidite, maleimide, haloacetyl, epoxide, alkylhalide, allyl halide, aldehyde, ketone, acylazide, anhydride, and iodoacetamide. Active esters include succinimidyl (NHS), hydroxybenzotriazolyl (HOBt) and pentafluorophenyl esters.

An NHS ester of a label reagent can be preformed, isolated, purified, and/or characterized, or it can be formed in situ and reacted with a nucleophilic group of a chimeric configurational oligonucleotide. A label carboxyl group can be activated by reacting with a combination of: (1) a carbodiimide reagent, e.g. dicyclohexylcarbodiimide-, diisopropylcarbodiimide, EDC (1-ethyl-3-(3-dimethylaminopropyl)carbodiim-ide); or a uronium reagent, e.g. TSTU (O—(N-Succinimidyl)-N,N,N′,N′-tetra-methyluronium tetrafluoroborate, HBTU (O-benzotriazol-1-yl)-N,N,N′,N′-tetr-amethyluronium hexafluorophosphate), or HATU (O-(7-azabenzotriazol-1-yl)-N-,N,N′,N′-tetramethyluronium hexafluorophosphate); and (2) an activator, such as HOBt (1-hydroxybenzotriazole) or HOAt (1-hydroxy-7-azabenzotriazo-le; and (3) N-hydroxysuccinimide to give the NHS ester.

One example of a non-nucleosidic phosphoramidite label reagent has the general formula VII, found in U.S. Patent Publication 2003/0198980, published Oct. 23, 2003 to Greenfield et al., page 14, paragraph 141. An alternative phosphoramidite labelling reagent is structure VIII, paragraph 143, of the same reference.

A phosphoramidite label reagent VII or VIII reacts with a hydroxyl group, e.g. 5′ terminal OH of a chimeric configurational oligonucleotide covalently attached to a solid support, under mild acid activation, e.g. tetrazole, to form an internucleotide phosphite group which is then oxidized to an internucleotide phosphate group. In some instances, the phosphoramidite label reagent contains functional groups that require protection either during the synthesis of the reagent or during its subsequent use to label a chimeric configurational oligonucleotide. The protecting group(s) used will depend upon the nature of the functional groups, and will be apparent to those having skill in the art (Greene, T. and Wuts, P. Protective Groups in Organic Synthesis, 2nd Ed., John Wiley & Sons, New York, 1991). The label will be attached at the 5′ terminus of the oligonucleotide, as a consequence of the common 3′ to 5′ direction of synthesis method with 5′-protected, 3′-phosphoramidite nucleosides. Alternatively, the 3′ terminus of an oligonucleotide can be labelled with a phosphoramidite label reagent when synthesis is conducted in the 5′ to 3′ direction with 3′-protected, 5′ phosphoramidite nucleosides, (Vinayak, U.S. Pat. No. 6,255,476).

Other phosphoramidite label reagents, both nucleosidic and non-nucleosidic, allow for labelling at other sites of a chimeric configurational oligonucleotide, e.g. 3′ terminus, nucleobase, internucleotide linkage, sugar. Labelling at the nucleobase, internucleotide linkage, and sugar sites allows for internal and multiple labelling.

As will be appreciated by one of skill in the art, donor or acceptor, marker or marker modifier moieties can be positioned on either the PNA segment or the L-DNA segment.

Fluorescent Interactions

In some embodiments, a reduced noise level for signal detection can be desirable. One method by which the presence or absence of a particular DM can be resolved in greater detail is through fluorescence resonance energy transfer (FRET). By associating the DM on the detection complex with a second DM or MM, near the location where the detection complex is or is supposed to form, one can create a system where the DM from the detection complex can emit light, and can do so in a manner that requires the particular pairing of the two DMs.

For FRET to occur, transfer of energy between donor and acceptor moieties requires that the moieties be close in space and that the emission spectrum of a donor(s) have substantial overlap with the absorption spectrum of the acceptor(s) (See: Yaron et al. Analytical Biochemistry, 95: 228-235 (1979) and particularly page 232, col. 1 through page 234, col. 1; additionally see pages 25 and 26 of the Ninth Edition of the Handbook of Fluorescent Probes and Research Products, which generally discloses FRET requirements, how to determine the Forster radius (R₀) and typical Forster radii for common FRET pairs, such as Fluorescein and tetramethylrhodamine, IAEDANS and Fluorescein, EDANS and Dabcyl, Fluorescein and Fluorescein, BODIPY FL and BODIPY FL, Fluorescein and QSY7 or QSY 9 dyes). It is also possible to use nanoparticles, such as Q-dot (quantum dots) as fluorophores to further increase the low detection limit (LOD). The universal dark quenchers, such as silver/golden nanoparticles, can also be used yielding even better quench efficiency.

Non-FRET interactions can also occur. In one embodiment, this is collision mediated (radiationless) energy transfer. This can occur between very closely associated donor and acceptor moieties whether or not the emission spectrum of a donor moiety(ies) has a substantial overlap with the absorption spectrum of the acceptor moiety(ies) (See: Yaron et al., Analytical Biochemistry, 95: 228-235 (1979) and particularly page 229, col. 1 through page 232, col. 1). This process is referred to as intramolecular collision since it is believed that quenching is caused by the direct contact of the donor and acceptor moieties (See: Yaron et al.). As demonstrated in molecular beacon experiments, the donor and acceptor moieties attached to analog probe complexes need not have a substantial overlap between the emission of the donor moieties and the absorbance of the acceptor moieties. Without intending to be bound to this hypothesis, this data suggested that collision and/or contact operates as the primary mode of quenching in analog probe complexes. In another embodiment, it is a change in environment around the fluorescent probe that results in the change in fluorescence; this may or may not directly be the acceptor moiety.

Nonfluorescent Signaling

As discussed above, not all signaling is achieved through a fluorescent signal. Many alternative examples are discussed herein and are known to one of skill in the art. Examples include MRI based techniques and binding based techniques, where the binding agent can either have a fluorescent moiety, catalyze a particular reaction, or similar signaling event, as well as other techniques.

Hybridization Conditions/Stringency:

Those of ordinary skill in the art of nucleic acid hybridization will recognize that factors commonly used to impose and/or control stringency of hybridization include formamide concentration (or other chemical denaturant reagent), salt concentration (i.e., ionic strength), hybridization temperature, detergent concentration, pH and the presence or absence of chaotropes. Optimal stringency for a probing nucleobase sequence/target sequence combination is often found by the well known technique of fixing several of the aforementioned stringency factors and then determining the effect of varying a factor. The same stringency factors can be modulated to control the stringency of hybridization of ID-tag complexes to target sequences, except that the hybridization of PNA sequences are fairly independent of ionic strength. Optimal stringency for an assay can be experimentally determined by examination of each stringency factor until the desired degree of discrimination is achieved.

Exemplary Applications for Using Some of the Various Embodiments:

Whether support bound or in solution, the methods, kits and compositions disclosed herein can be useful for the rapid, sensitive, reliable and versatile detection of target sequences which are particular to organisms which might be found in food, beverages, water, pharmaceutical products, personal care products, dairy products and/or environmental samples. Preferred beverages include soda, bottled water, fruit juice, beer, wine, or liquor products. The methods, kits and compositions disclosed herein will be particularly useful for the analysis of raw materials, equipment, products and/or processes used to manufacture or store food, beverages, water, pharmaceutical products, personal care products, dairy products and/or environmental samples.

Whether support bound or in solution, the methods, kits and compositions are also particularly useful for the rapid, sensitive, reliable and versatile detection of target sequences which are particular to organisms which might be found in clinical environments. Consequently, the methods, kits and compositions disclosed herein will be useful for the analysis of clinical specimens, equipment, fixtures, and/or products used to treat humans and/or animals. For example, assays can be used to detect a target sequence that is specific for a genetically based disease and/or is specific for a predisposition to a genetically based disease. Non-limiting examples of diseases include, beta-Thalassemia, sickle cell anemia, Factor-V Leiden, cystic fibrosis and cancer related targets such as p53, p10, BRC-1 and BRC-2. The target sequence can be related to a chromosomal DNA, wherein the detection, identification and/or quantitation of the target sequence can be used in relation to forensic techniques such as prenatal screening, paternity testing, identity confirmation or crime investigation. The target sequence can be particularly short pieces of nucleic acids or analogs thereof. For example, the target sequence can be siRNA, miRNA, or other such relatively short sequences. In one embodiment, sequences less than 100 nucleotides are contemplated as the target sequence. For example, lengths of 100-90, 90-70, 70-50, 50-40, 40-30, 30-25, 25-20, 20-15, 15-10, 10-05, including lesser lengths, are contemplated.

In some embodiments, the above sizes can also be used for detection of segments of the above sizes, rather than sequences of the above sizes.

In this application, the use of the singular can include the plural unless specifically stated otherwise or unless, as will be understood by one of skill in the art in light of the present disclosure, the singular is the only functional embodiment. Thus, for example, “a” can mean more than one, and “one embodiment” can mean that the description applies to multiple embodiments. Additionally, in this application, “and/or” denotes that both the inclusive meaning of “and” and, alternatively, the exclusive meaning of “or” applies to the list. Thus, the listing should be read to include all possible combinations of the items of the list and to also include each item, exclusively, from the other items. The addition of this term is not meant to denote any particular meaning to the use of the terms “and” or “or” alone. The meaning of such terms will be evident to one of skill in the art upon reading the particular disclosure.

### Incorporation by Reference

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

### Equivalents

The foregoing description and Examples detail certain preferred embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

What is claimed is:

1. An ID-tag complex comprising:
a probe section comprising a probe sequence, an ID-tag sequence, and a detectable marker, wherein the probe sequence is connected to the ID-tag sequence and the detectable marker; and
a probe complement section comprising a probe complement sequence connected to a first coupling molecule, wherein at least a portion of said probe sequence and said probe complement sequence are configured to hybridize to one another, and wherein the length of the probe sequence is at least 1 nucleotide greater than the length of the probe complement sequence.

2. The ID-tag complex of claim 1, wherein the detectable marker is connected to an end of the probe sequence and the ID-tag sequence is connected an opposite end of the probe sequence

3. The ID-tag complex of claim 1, wherein the probe section comprises an analog probe sequence.

4. The ID-tag complex of claim 3, wherein the analog probe sequence comprises L-DNA.

5. The ID-tag complex of claim 3, wherein the analog probe sequence comprises PNA.

6. The ID-tag complex of claim 1, wherein the probe sequence can bind to RNA.

7. The ID-tag complex of claim 6, wherein the probe sequence can bind to a RNA target sequence.

8. The ID-tag complex of claim 1, wherein the probe sequence can bind to miRNA.

9. The ID-tag complex of claim 8, wherein the probe sequence is complementary to a miRNA target sequence.

10. The ID-tag complex of claim 1, wherein the probe sequence is 5-10 bases longer than the probe complement sequence.

11. The ID-tag complex of claim 1, wherein the extra length of the probe sequence compared to the probe complement sequence is as an overhang on one end of the probe sequence.

12. The ID-tag complex of claim 11, wherein the probe sequence has an overhang of 6 or 7 bases over the probe complement sequence.

13. The ID-tag complex of claim 1, wherein the probe sequence comprises 2′ O-methyl RNA.

14. The ID-tag complex of claim 1, wherein the first coupling molecule comprises biotin.

15. The ID-tag complex of claim 1, wherein the detectable marker comprises DIG.

16. The ID-tag complex of claim 1, wherein the ID-tag sequence comprises an analog nucleotide.

17. The ID-tag complex of claim 16, wherein the ID-tag sequence comprises L-DNA.

18. The ID-tag complex of claim 1, further comprising a linker between the probe sequence and the ID-tag sequence.

19. An ID-tag detection complex comprising:
a probe section, said probe section comprising a first ID-tag sequence connected to a probe sequence, wherein said probe sequence is also connected to a detectable marker;
a target section comprising a target sequence, wherein said target sequence is hybridized to the probe sequence; and
an ID-tag detection section comprising a second ID-tag sequence that is hybridized to said first ID-tag sequence

20. The ID-tag detection complex of claim 19, wherein said ID-tag detection section is located at a first location in an array system.

21. A method of detecting a target segment in a sample, said method comprising:
contacting an ID-tag complex with a sample such that the probe sequence hybridizes to a target sequence in the sample, wherein said ID-tag complex comprises 1) a probe section comprising a probe sequence, an ID-tag sequence, and a detectable marker, wherein the probe sequence is connected to the ID-tag sequence and is further connected to the detectable marker, and the ID-tag complex further comprises 2) a probe complement section comprising a probe complement sequence connected to a first coupling molecule, wherein at least a portion of said probe sequence and said probe complement sequence are configured to hybridize to one another, and wherein the length of the probe sequence is at least 1 nucleotide greater than the length of the probe complement sequence;
contacting a second coupling molecule to the sample so that the second coupling molecule can bind to substantially all of the first coupling molecule;
removing substantially all of the second coupling molecule; and
detecting the detectable marker in the sample, thereby detecting a target segment in the sample.

22. The method of claim 21, further comprising the steps of:
adding the sample to an array, said array comprising an ID-tag detection sequence that is complementary to the ID-tag sequence of the ID-tag complex at a first position; and
detecting the presence of the detectable marker at the first position, thereby detecting the presence the target segment in the sample.

23. The method of claim 22, wherein the target is RNA.

24. The method of claim 23, wherein the target is miRNA.

25. The method of claim 22, wherein the first coupling molecule is biotin.

26. The method of claim 22, wherein the second coupling molecule is streptavidin.

27. The method of claim 22, wherein the array comprises more than one ID-tag detection sequence.

28. The method of claim 27, wherein the array comprises an ID-tag detection sequence that is specific for the same ID-tag sequence.

29. The method of claim 27, wherein the array comprises ID-tag detection sequences that are specific for different ID-tag target sequences.

30. The method of claim 22, wherein 1) the array comprises at least two different ID-tag detection sequences and 2) there are at least parts of two different ID-tag probe complexes that are added to a sample, wherein at least one of the ID-tag probe complexes has a probe sequence that is different from probe sequence in a different ID-tag probe complex.

31. The method of claim 22, wherein at least three different ID-tag sequences comprising three different ID-tag probes are used.

32. An ID-tag complex kit comprising:
an ID-tag complex comprising 1) a probe section comprising a detectable marker, an ID-tag sequence, and a probe sequence; and 2) a probe complement section comprising a first coupling molecule attached to a probe complement sequence, wherein at least a portion of said probe complement sequence and said probe sequence are capable of hybridizing.

33. The kit of claim 32, further comprising a second coupling molecule;

34. The kit of claim 33, further comprising an array, said array comprising an ID-tag detection sequence, wherein said ID-tag detection sequence can hybridize to said first ID-tag sequence.

35. The kit of claim 32, further comprising an RNase inhibitor.

36. The kit of claim 32, further comprising a means for isolating miRNA.
